SEQUENCE LISTING (32 sequences)

SEQ ID NO: 1 - protein, 6 aa, Artificial Sequence
  L1 Linker
ASTKGP

SEQ ID NO: 2 - protein, 13 aa, Artificial Sequence
  L1 Linker
ASTKGPSVFPLAP

SEQ ID NO: 3 - protein, 10 aa, Artificial Sequence
  L1 Linker
GGGGSGGGGS

SEQ ID NO: 4 - protein, 5 aa, Artificial Sequence
  L2 Linker
TVAAP

SEQ ID NO: 5 - protein, 12 aa, Artificial Sequence
  L2 Linker
TVAAPSVFIFPP

SEQ ID NO: 6 - protein, 9 aa, Artificial Sequence
  L2 Linker
GGSGGGGSG

SEQ ID NO: 7 - protein, 5 aa, Artificial Sequence
  Inter Disulfide Domain IDD1
EPKSC

SEQ ID NO: 8 - protein, 4 aa, Artificial Sequence
  Inter Disulfide Domain IDD2
RGEC

SEQ ID NO: 9 - protein, 250 aa, Artificial Sequence
  VEGF TNF SL RGEC
QVTLKESGPTMVKPTQTLTLTCTFSGFSLSTSGVAVGWIRQPPGQALEWLALIYWDNDKRYSPSLKNRLTVAKDTSKSQVVLTMTNMDPMDTATYYCAHGDGWLFDFWGQGTLVTVSSASTKGPEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSEPKSC

SEQ ID NO: 10 - protein, 477 aa, Artificial Sequence
  VEGF-TNF-SL-RGEC-Fc
QVTLKESGPTMVKPTQTLTLTCTFSGFSLSTSGVAVGWIRQPPGQALEWLALIYWDNDKRYSPSLKNRLTVAKDTSKSQVVLTMTNMDPMDTATYYCAHGDGWLFDFWGQGTLVTVSSASTKGPEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 228 aa, Artificial Sequence
  VEGF-TNF-SL-RGEC LC
QPVLTQPPSVSGAPGQRVTISCTGSNSNIGAGHDVHWYQQLPGAAPKVVIYGNTNRASGVPERFSGSKSATSASLAITGLQAEDEADYYCQSYDNSLSGYVFGTGTKVTVLRTVAAPDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGEC

SEQ ID NO: 12 - protein, 245 aa, Artificial Sequence
  VEGF-TNF-SL no S-S Bridge
QVTLKESGPTMVKPTQTLTLTCTFSGFSLSTSGVAVGWIRQPPGQALEWLALIYWDNDKRYSPSLKNRLTVAKDTSKSQVVLTMTNMDPMDTATYYCAHGDGWLFDFWGQGTLVTVSSASTKGPEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 13 - protein, 472 aa, Artificial Sequence
  VEGF-TNF-SL no S-S Bridge-Fc
QVTLKESGPTMVKPTQTLTLTCTFSGFSLSTSGVAVGWIRQPPGQALEWLALIYWDNDKRYSPSLKNRLTVAKDTSKSQVVLTMTNMDPMDTATYYCAHGDGWLFDFWGQGTLVTVSSASTKGPEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 224 aa, Artificial Sequence
  VEGF-TNF-SL no S-S Bridge LC
QPVLTQPPSVSGAPGQRVTISCTGSNSNIGAGHDVHWYQQLPGAAPKVVIYGNTNRASGVPERFSGSKSATSASLAITGLQAEDEADYYCQSYDNSLSGYVFGTGTKVTVLRTVAAPDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

SEQ ID NO: 15 - protein, 273 aa, Artificial Sequence
  TNF-IL17-SL
MGWSYIILFLVATAADVHSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSS

SEQ ID NO: 16 - protein, 500 aa, Artificial Sequence
  TNF-IL17-SL-Fc
MGWSYIILFLVATAADVHSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 222 aa, Artificial Sequence
  TNF-IL17-SL LC
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 18 - protein, 273 aa, Artificial Sequence
  eTNF-IL17-SL
MGWSYIILFLVATAADVHSEVQLVESGGGLVQPGRSLRLSCAASGFTFDDYAMHWVRQAPGKCLEWVSAITWNSGHIDYADSVEGRFTISRDNAKNSLYLQMNSLRAEDTAVYYCAKVSYLSTASSLDYWGQGTLVTVSSASTKGPEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSS

SEQ ID NO: 19 - protein, 500 aa, Artificial Sequence
  eTNF-IL17-SL-Fc
MGWSYIILFLVATAADVHSEVQLVESGGGLVQPGRSLRLSCAASGFTFDDYAMHWVRQAPGKCLEWVSAITWNSGHIDYADSVEGRFTISRDNAKNSLYLQMNSLRAEDTAVYYCAKVSYLSTASSLDYWGQGTLVTVSSASTKGPEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 222 aa, Artificial Sequence
  eTNF-IL17-SL LC
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGCGTKVEIKRTVAAPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 21 - protein, 280 aa, Artificial Sequence
  eTNF-IL17-LL
MGWSYIILFLVATAADVHSEVQLVESGGGLVQPGRSLRLSCAASGFTFDDYAMHWVRQAPGKCLEWVSAITWNSGHIDYADSVEGRFTISRDNAKNSLYLQMNSLRAEDTAVYYCAKVSYLSTASSLDYWGQGTLVTVSSASTKGPSVFPLAPEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSS

SEQ ID NO: 22 - protein, 507 aa, Artificial Sequence
  eTNF-IL17-LL-Fc
MGWSYIILFLVATAADVHSEVQLVESGGGLVQPGRSLRLSCAASGFTFDDYAMHWVRQAPGKCLEWVSAITWNSGHIDYADSVEGRFTISRDNAKNSLYLQMNSLRAEDTAVYYCAKVSYLSTASSLDYWGQGTLVTVSSASTKGPSVFPLAPEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 229 aa, Artificial Sequence
  eTNF-IL17-LL LC
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGCGTKVEIKRTVAAPSVFIFPPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 24 - protein, 277 aa, Artificial Sequence
  eTNF-IL17-G4S
MGWSYIILFLVATAADVHSEVQLVESGGGLVQPGRSLRLSCAASGFTFDDYAMHWVRQAPGKCLEWVSAITWNSGHIDYADSVEGRFTISRDNAKNSLYLQMNSLRAEDTAVYYCAKVSYLSTASSLDYWGQGTLVTVSSGGGGSGGGGSEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSS

SEQ ID NO: 25 - protein, 504 aa, Artificial Sequence
  eTNF-IL17-G4S-Fc
MGWSYIILFLVATAADVHSEVQLVESGGGLVQPGRSLRLSCAASGFTFDDYAMHWVRQAPGKCLEWVSAITWNSGHIDYADSVEGRFTISRDNAKNSLYLQMNSLRAEDTAVYYCAKVSYLSTASSLDYWGQGTLVTVSSGGGGSGGGGSEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 226 aa, Artificial Sequence
  eTNF-IL17-G4S LC
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGCGTKVEIKRGGSGGGGSGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 27 - protein, 277 aa, Artificial Sequence
  TNF-eIL17-G4S
MGWSYIILFLVATAADVHSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSEVQLVESGGGLVQPGGSLRLSCAASGFTFSNYWMNWVRQAPGKCLEWVAAINQDGSEKYYVGSVKGRFTISRDNAKNSLYLQMNSLRVEDTAVYYCVRDYYDILTDYYIHYWYFDLWGRGTLVTVSS

SEQ ID NO: 28 - protein, 504 aa, Artificial Sequence
  TNF-eIL17-G4S-Fc
MGWSYIILFLVATAADVHSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSEVQLVESGGGLVQPGGSLRLSCAASGFTFSNYWMNWVRQAPGKCLEWVAAINQDGSEKYYVGSVKGRFTISRDNAKNSLYLQMNSLRVEDTAVYYCVRDYYDILTDYYIHYWYFDLWGRGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 226 aa, Artificial Sequence
  TNF-eIL17-G4S LC
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGSGGGGSGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGCGTRLEIKR

SEQ ID NO: 30 - protein, 257 aa, Artificial Sequence
  eABT122_Fv2mab
EVQLVESGGGLVQPGRSLRLSCAASGFTFDDYAMHWVRQAPGKCLEWVSAITWNSGHIDYADSVEGRFTISRDNAKNSLYLQMNSLRAEDTAVYYCAKVSYLSTASSLDYWGQGTLVTVSSGGGGSGGGGSEVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWNGYYSTHDFDSWGQGTTVTVSS

SEQ ID NO: 31 - protein, 484 aa, Artificial Sequence
  eABT122_Fv2mab-Fc
EVQLVESGGGLVQPGRSLRLSCAASGFTFDDYAMHWVRQAPGKCLEWVSAITWNSGHIDYADSVEGRFTISRDNAKNSLYLQMNSLRAEDTAVYYCAKVSYLSTASSLDYWGQGTLVTVSSGGGGSGGGGSEVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWNGYYSTHDFDSWGQGTTVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 225 aa, Artificial Sequence
  eABT122_Fv2mab LC
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGCGTKVEIKRGGSGGGGSGEIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKR

(19) United States
(12) Patent Application Publication
(10) Pub. No.: US 2019/0389973 A1
(43) Pub. Date: Dec. 26, 2019

(54) NOVEL MULTI-SPECIFIC BINDING PROTEINS

(71) Applicant: Y-BIOLOGICS INC., Daejeon (KR)

(72) Inventors: Seil JANG, Daejeon (KR); Young Woo PARK, Daejeon (KR); Bum-chan PARK, Daejeon (KR)

(21) Appl. No.: 16/483,433

(22) PCT Filed: Apr. 18, 2017
(86) PCT No.: PCT/KR2017/004154

§ 371 Date: Aug. 4, 2019

(30) Foreign Application Priority Data
Feb. 20, 2017 (KR) 10-2017-0022270

(51) Int. Cl. C07K 16/46; C07K 16/24; C07K 16/22

(52) U.S. Cl. CPC C07K 16/468; C07K 2317/64; C07K 16/244; C07K 16/22; C07K 2317/56; C07K 2317/524; C07K 2317/526; C07K 2317/31; C07K 2317/624; C07K 2317/567; C07K 2317/35; C07K 2317/92; C07K 2317/94; C07K 2317/76; C07K 16/241

(57) ABSTRACT

The present disclosure relates to a novel multi-specific binding protein, specifically, a novel multi-specific binding protein prepared by supplementing drawbacks of various conventionally disclosed multi-specific binding proteins, for example, a bispecific binding protein, more specifically, a novel multi-specific binding protein including a polypeptide, wherein heavy chain CH1 domain and CL domain of a antibody constant region are not included but a heavy chain variable region and/or a light chain variable region are consecutively linked in the polypeptide.

### CROSS-REFERENCE TO RELATED APPLICATIONS

This application is a U.S. national phase under the provisions of 35 U.S.C. § 371 of International Patent Application No. PCT/KR17/04154 filed Apr. 18, 2017, which in turn claims priority of Korea, Republic of Patent Application No. 10-2017-0022270 filed Feb. 20, 2017. The disclosures of such international patent application and Korea, Republic of priority patent application are hereby incorporated herein by reference in their respective entireties, for all purposes.

### TECHNICAL FIELD

The present disclosure relates to a novel multi-specific binding protein, specifically, a novel multi-specific binding protein prepared by supplementing drawbacks of various conventionally disclosed multi-specific binding proteins, for example, a bispecific binding protein, more specifically, a novel multi-specific binding protein including a polypeptide, wherein heavy chain CH1 domain and CL domain of an antibody constant region are not included but a heavy chain variable region and/or a light chain variable region are consecutively linked in the polypeptide.

### DESCRIPTION OF THE RELATED ART

As the production of gene recombinant proteins and human antibody library screening based on phage display technology become easy, the development of therapeutic agents using an antibody has been activated. Therapeutic agents using an antibody act on the basis of the unique immune system of human body by using the specific antigen-antibody binding, and thus the toxicity of therapeutic agents using an antibody is lower than that of conventional chemical therapeutic agents. Therefore, antibody-based therapeutic agents currently developed and used clinically are actively used in various fields such as anti-cancer agents, autoimmune and inflammatory diseases, infectious diseases, and organ transplantation.

As various causes and mechanisms of a single indication are recently found, methods of developing a therapeutic agent are shifting from a single-target approach to a multi-target approach. Various studies have been conducted for decades in the development of antibody-based therapeutic agents to add a multi-specific mechanism to a single-specific antibody to enable specific binding to two or more antigenic proteins. The first bispecific antibody was developed by using Quadroma Technology (Milstein, C., & Cuello, A. C. (1984). Hybrid hybridomas and the production of bi-specific monoclonal antibodies. Immunology Today, 5(10), 299-304. doi:10.1016/0167-5699(84)90155-5), wherein a bispecific antibody was developed through cell fusion of hybridoma strain lines expressing different antibodies. Various types of bispecific antibodies have been developed, as the production of gene recombinant proteins using animal cells and the structure-based protein engineering technology have been drastically advanced. Representative examples of monovalent bispecific antibody include a two-in-one bispecific antibody (Bostrom, J., Yu, S.-F., Kan, D., Appleton, B. A., Lee, C. V., Billeci, K., et al. (2009). Mutants of the antibody herceptin that interact with HER2 and VEGF at the antigen binding site. Science, 323(5921), 1610-1614. doi:10.1126/science.1165480) prepared by applying knobs-into-holes design to the CH3 domain of an antibody to enable binding to two different kinds of antigens by inducing effective heterodimerization of heavy chains and CrossMab (Schaefer, W., Regula, J. T., Bahner, M., Schanzer, J., Croasdale, R., Ma, H., et al. (2011). Immunoglobulin domain crossover as a generic approach for the production of bispecific IgG antibodies. Proceedings of the National Academy of Sciences of the United States of America, 108(27), 11187-11192. doi:10.1073/pnas.1019002108) prepared on the basis of heavy chain heterodimerization by inducing selective heterodimerization in one light chain through a crossover between the light chain VL or CL domain and the heavy chain VH or CH1 domain. However, with regard to these heterodimerization-based bispecific antibodies, the homogenous types and heterogeneous types simultaneously existing in an expression medium are difficult to distinguish in the production of recombinant proteins using animal cells to obtain bispecific antibodies, and the activity is low in comparison with conventional parental bivalent antibodies.

In addition, mutation introduced for selective heterodimerization of heavy chain and light chain may cause a stability problem, such as immunogenicity. Recently, development of dual antibody is actively performed on the basis of dual-variable-domain immunoglobulin (DVD-Ig, AbbVie, USA) relatively free from the CMC (Chemistry, Manufacturing, Control) problems involved in the polypeptide issues of various combinations of the Genentech two-in-one antibody and Roche CrossMab. Representative targets of the DVD-Ig format application include IL-1α/IL-1β, IL-12/IL-18, VEGF/osteopontin, and TNF-α/IL-17, and development and clinical studies of the therapeutic agents are conducted with a strategy of simultaneously neutralizing target proteins (Wu, C., Ying, H., Grinnell, C., Bryant, S., Miller, R., Clabbers, A., et al. (2007). Simultaneous targeting of multiple disease mediators by a dual-variable-domain immunoglobulin. Nature Biotechnology, 25(11), 1290-1297. doi:10.1038/nbt1345).

In the DVD-Ig format, a variable region of an antibody that may be combined to another antigen is consecutively linked with an N-terminal of a conventional IgG antibody variable region. Therefore, the molecular weight of a DVD-Ig is about 200 kDa, as the molecular weight is increased as much as the molecular weight of the variable region linked through a linker to add a function. In the development of protein and antibody-based medical product, an antibody or a protein medical product having a smaller molecular weight may provide more effective penetration to cancer cells or lesions and reach a high concentration in the blood within a shorter period of time to give a higher molar effect with a same weight when administered in a clinical experiment or as a therapeutic agent.

In this technical background, the inventors of the present application have made efforts to develop a new format of multi-specific multivalent binding protein and prepared a binding protein having a molecular weight of 150 kDa the same as that of conventional antibodies (tetravalent bispecific Fv2mab in one example) by substituting the CH1 domain and CL domain of heavy chain and light chain constant regions with VH and VL, wherein the VH and VL are the variable domains needed for binding to a secondary antigen, and the drawbacks of conventional technologies described above were supplemented and the inhibition of activity through the simultaneous binding to two or more targets was verified as purposed by the present invention.

### SUMMARY

The purpose of the present invention is to develop a novel binding protein (for example, bispecific antibody Fv2mab) platform that may effectively blocking and inhibiting signal transduction and biological activity of various protein molecules causing various indications, such as autoimmune diseases, angiogenesis, and cancer cell growth (for example, cytokine, chemockine, etc). Another purpose of the present invention is to provide a conjugates including the binding proteins. To accomplish the purposes, the prevent invention provides a binding protein comprising a first polypeptide including VH1-[(L1)a-VH2]m-Xb and a second polypeptide including VL1-[(L2)c-VL2]n, wherein VH1 or VH2 of the first polypeptide is a heavy chain variable region respectively including a same or different antigen-binding region or a variant thereof, VL1 or VL2 of the second polypeptide is a light change variable region respectively including a same or different antigen-binding region or a variant thereof, the second polypeptide lack CL, which means that the second polypeptide does not include CL, L1 of the first polypeptide is a linker existing between VH1 and VH2 to link VH1 and VH2 consecutively, L2 of the second polypeptide is a linker existing between VL1 and VL2 to link VL1 and VL2 consecutively, X of the first polypeptide is a Fc including CH2 and CH3 domains and excluding CH1, a, b, and c are respectively 0 or 1, and m and n are respectively an integer between 1 and 10. The prevent invention also provides a binding protein including a first polypeptide consisting of VH1-[(L1)a-VH2-(IDD1)b]m-Xc and a second polypeptide consisting of VL1-[(L2)d-VL2-(IDD2)e]n, wherein VH1 or VH2 of the first polypeptide is a heavy chain variable region respectively including a same or different antigen-binding region or a variant thereof, VL1 or VL2 of the second polypeptide is a light change variable region respectively including a same or different antigen-binding region or a variant thereof, L1 of the first polypeptide is a linker existing between VH1 and VH2 to link VH1 and VH2 consecutively, L2 of the second polypeptide is a linker existing between VL1 and VL2 to link VL1 and VL2 consecutively, IDD1 of the first polypeptide is an inter-disulfide domain for generating a disulfide bridge to a light chain, IDD2 of the second polypeptide is an inter-disulfide domain for generating a disulfide bridge to a heavy chain, and X of the first polypeptide is a Fc including CH2 and CH3 domains, a, b, c, d, and e are respectively 0 or 1, and m and n are respectively an integer between 1 and 10.

The present invention also provides an antibody including a plurality of binding proteins.

The present invention also provides a conjugate including the binding protein.

### DESCRIPTION OF DRAWINGS

The patent or application file contains at least one drawing executed in color. Copies of this patent or patent application publication with color drawing(s) will be provided by the Office upon request and payment of the necessary fee.

The above and other aspects, features and other advantages of the present disclosure will be more clearly understood from the following detailed description taken in conjunction with the accompanying drawings, in which:

FIG. 1a shows the brief structure of the bispecific antibody Fv2mab.

FIG. 1b shows the structure of engineered Fv2mab prepared by introducing an inter-disulfide bridge. The part where a cysteine disulfide bond may be introduced is RGEC amino acids of a light chain and EPKSC amino acids of a heavy chain introduced to a C-terminal of an outer variable domain and an inner variable domain.

FIG. 2 shows the secondary structure of Fv2mab prepared by applying a linker and inter-disulfide bridge engineering.

FIG. 3 shows the length of the inter-disulfide domain (IDD) for RGEC engineering.

FIG. 4 shows the expression cassette of Fv2mabs cloned to pFE vector.

FIG. 5 shows the result of the Fv2mab expression verification using SDS-PAGE and the result of protein A pull down assay.

FIG. 6 shows the SDS-PAGE result verifying that the QC for the assembly and secretion of Fv2mab is controlled by a BiP-dependen manner (structure selection).

FIG. 7 shows the result verifying the efficiency of inter-disulfide bridge of Fv2mab prepared by cysteine engineering.

FIG. 8 shows the native PAGE result verifying the conformation of Fv2mab (pH 7.4, PBS) without an inter-disulfide bridge between a heavy chain and a light chain.

FIG. 9 shows the two-dimensional SDS-PAGE results of Fv2mab prepared only by binding of VH and VL.

FIG. 10 shows the conformation of engineered Fv2mab clones verified by using Agilent bioanalyzer 2100 and the result verifying the purity of H44L100 cysteine-engineered Fv2mab.

FIG. 11 shows the result verifying the purity of HUMIRA, SECUKINUMAB, Fv2mab_TNF-α-IL-17-SL, and Fv2mab_eTNF-α-IL-17-SL by using SEC-HPLC.

FIG. 12 shows the result verifying the stability of Fv2mabs (engineered or not engineered antibody) in human serum.

FIG. 13 shows the result comparing the binding affinity of Fv2mabs to VEGFA of an external Fv.

FIG. 14 shows the result comparing the binding affinity of Fv2mabs to TNF-α of an internal Fv.

FIG. 15 shows the result comparing the binding affinity of Fv2mabs to TNF-α of an external Fv.

FIG. 16 shows the result comparing the binding affinity of Fv2mabs to IL-17 of an internal Fv.

FIG. 17 shows the result comparing the IC50 values of SECUKINUMAB, DVD-Ig (ABT-122), and Fv2mabs with regard to IL-17.

FIG. 18 shows the result comparing the Kd values by using Surface Plasmon Resonance (SPR).

FIG. 19 shows the result comparing the TNF-α blockade efficacy of Fv2mabs measured by an in vitro cell-based assay.

FIG. 20 shows the result comparing the IL-17 blockade efficacy of Fv2mabs measured by an in vitro cell-based assay.

FIG. 21 shows the result comparing the binding affinity of Mono/dimeric linked Fv (number of linked Fv).

FIG. 22 shows the result comparing the antigen-binding affinity of Fv2mab depending on linkers (short, long, G4S).

FIG. 23 shows the result comparing the in vitro efficacy (cell-based assay) of Fv2mab depending on linkers.

FIG. 24 shows the result comparing the antigen-binding affinity depending on bispecific antibody formats (DVD-Ig vs. Fv2mab).

FIG. 25 shows the result comparing the TNF-α/IL-17 blockade efficacy of DVD-Ig and Fv2mab (cell-based assay).

FIG. 26 shows the degree of blockade of signal transduction simultaneously induced by TNF-α and IL-17A, wherein the degree of blockade of signal transduction was measured by using HT-29 cells (comparison of blockage efficacy between bispecific antibody and monospecific antibody).

### DETAILED DESCRIPTION OF THE PREFERRED EMBODIMENT

Unless defined otherwise, all technical and scientific terms used herein have the same meaning as commonly understood by one of ordinary skill in the art to which the invention pertains. Generally, the nomenclature used herein and the experimental methods which will be described later are those well known and commonly employed in the art.

One aspect of the present invention provides a binding protein comprising a first polypeptide of VH1-[(L1)a-VH2]m-Xb and a second polypeptide of VL1-[(L2)c-VL2]n, wherein VH1 or VH2 of the first polypeptide is a heavy chain variable region respectively comprising a same or different antigen-binding region, VL1 or VL2 of the second polypeptide is a light change variable region respectively comprising a same or different antigen-binding region, the second polypeptide lacks CL, L1 of the first polypeptide is a linker existing between VH1 and VH2 to link VH1 and VH2 consecutively, L2 of the second polypeptide is a linker existing between VL1 and VL2 to link VL1 and VL2 consecutively, X of the first polypeptide is a Fc comprising CH2 and CH3 domains and excluding CH1, a, b, and c are respectively 0 or 1, and m and n are respectively an integer between 1 and 10.

It is generally known that the expression and secretion of an antibody is determined by the assembly of a heavy chain and a light chain in endoplasmic reticulum (ER), wherein a binding immunoglobulin protein (BiP), a HSP70 chaperon, and a heavy chain CH1 domain play the role (Feige, M. J., Groscurth, S., Marcinowski, M., Shimizu, Y., Kessler, H., Hendershot, L. M., & Buchner, J. (2009). An Unfolded CH1 Domain Controls the Assembly and Secretion of IgG Antibodies. Molecular Cell, 34(5), 569-579. doi:10.1016/j.molce1.2009.04.028). However, many experiments performed by the inventor of the present application has shown that the BiP-dependent antibody assembly occurring in ER is not performed exclusively by CH1 and CL of an antibody but VH and VL of an antibody also play a role in antibody assembly in a BiP-dependent manner. This indicates that antibody assembly is determined not by the presence or absence of CH1 and CL domains but by both VL and CL domains, wherein the VL and CL domains are light chain folding partners that may effectively release BiP bound to heavy chain VH and CH1.

In other words, antibody assembly is controlled by a mechanism wherein the BiP bound to VH is released only by VL and the BiP bound to CH1 is released only by CL. Therefore, the heavy chain CH1 domain and light chain CL domain of an antibody are not the necessary components of an antibody, and, instead, a heavy chain VH and a light chain VL to which a secondary antigen may be bound were respectively and consecutively linked to prepare a multivalent bispecific antibody format. As a secondary variable region is introduced instead of the CH1 and CL domains, an H2L2 format bispecific antibody Fv2mab has a size of about 150 kDa similar to the molecular weight of a general antibody. In addition, since the size of Fv2mab (150 kDa) is relatively small in comparison with the DVD-Ig format (approximately 200 kDa) including CH1 and CL domains, Fv2mab may have an advantage in the development of an antibody-based medical product. The bispecific antibody Fv2mab productivity was found to be about 100 to 200 mg/L as measured by using an HEK 293 transient expression system possessed by the present applicant, wherein the bispecific antibody Fv2mab productivity was relatively higher than that of DVD-Ig.

In addition, in comparison with Diabody (Holliger, P., Prospero, T., & Winter, G. (1993). “Diabodies”: small bivalent and bispecific antibody fragments. Proceedings of the National Academy of Sciences, 90(14), 6444-6448) using a prokaryotic expression system, such as E. coli, and other ScFv-based bispecific antibody formats, Fv2mab using a mammalian expression system is superior in aspects of productivity, quality, and CMC (Chemistry, Manufacturing, Control).

The term “binding protein” used herein refers to a protein that may be specifically bound to a target and means an immunoglobulin molecule, an antibody, a fragment thereof, a variant thereof, or a modification thereof. The binding protein according to the present invention includes a first polypeptide including VH1-(L1)a-VH2-Xb and a second polypeptide including VL1-(L2)c-VL2, and does not include the heavy chain first constant region (CH1) and the light chain constant region of a Fab fragment. In some cases, the binding protein according to the present invention includes a first polypeptide consisting of VH1-[(L1)a-VH2-(IDD1)b]m-Xc and a second polypeptide consisting of VL1-[(L2)d-VL2-(IDD2)e]n. The VH1 or VH2 of the first polypeptide is a heavy chain variable region respectively including a same or different antigen-binding region or a variant thereof, VL1 or VL2 of the second polypeptide is a light chain variable region respectively including a same or different antigen-binding region or a variant thereof, L1 of the first polypeptide is a linker existing between VH1 and VH2 to link VH1 and VH2 consecutively, L2 of the second polypeptide is a linker existing between VL1 and VL2 to link VL1 and VL2 consecutively, IDD1 of the first polypeptide is an inter-disulfide domain for generating a disulfide bridge to a light chain, IDD2 of the second polypeptide is an inter-disulfide domain for generating a disulfide bridge to a heavy chain, and X of the first polypeptide is a Fc including CH2 and CH3 domains, a, b, c, d, and e are respectively 0 or 1, and m and n are respectively an integer between 1 and 10.

A Fab fragment has a structure having a variable region of a light chain and a heavy chain and a constant region of a light chain and a first constant region of a heavy chain (CH1) and includes one antigen-binding site. A Fab′ fragment is different from Fab because a Fab′ fragment has a hinge region including one or more cysteine residue. F(ab′)₂ is formed when cysteine residues in the hinge region of Fab′ form a disulfide bond. Fv is a minimum antibody fragment having only a heavy chain variable region and a light chain variable region. A heavy chain variable region and a light chain variable region are bound to each other by a noncovalent bond in a two-chain Fv. In a single-chain Fv (scFv), a heavy chain variable region and a light chain variable region are generally linked by a covalent bond through a peptide linker or directly linked at the C-terminals to form a dimer structure as in a two-chain Fv. These antibody fragments may be obtained by using a protein hydrolysis enzyme (For example, an entire antibody may be restriction-fragmented by using papain to obtain Fab or fragmented by using pepsin to obtain F(ab′)₂ fragments.) or prepared through gene recombinant technology.

The term “polypeptide” used herein refers to an arbitrary polymer chain of amino acids. The terms “peptide” and “protein” may be used interchangeably with the term “polypeptide,” wherein the terms “peptide” and “protein” also refer to a polymer chain of amino acids. A polypeptide includes a natural or synthetic protein, a protein fragment, and a polypeptide analogue of a protein sequence. A polypeptide may be a monomer or a polymer.

With regard to an interaction of an antibody, polypeptide, protein, or peptide, the term “specific binding” or “specifically binding” used herein means that the interaction is dependent on the presence of a specific structure of a chemical species (for example, an antigen determinant or epitope). For example, an antibody generally recognizes and binds to a specific protein structure, rather than a protein. When an antibody is specific to epitope “A,” in a reaction including a labeled “A” and the antibody, the presence of molecules including epitope A will reduce the amount of labeled A bound to the antibody.

The term “antibody” used herein refers to an arbitrary functional fragment, mutant or variant, modification or derivative having an arbitrary immunoglobulin (Ig) molecule and an epitope binding characteristic necessary to an Ig molecule, wherein an Ig molecule consists of four polypeptide chains, including two heavy chains (H) and two light chains (L). Specific examples of the mutant or variant, modification or derivative are described below but not limited thereto.

In a complete antibody, each heavy chain consists of a heavy chain variable region (expressed as HCVR or VH) and a heavy chain constant region. The heavy chain constant region consists of three domains, wherein the three domains are expressed as CH1, CH2, and CH3. A light chain constant region consists of one domain, wherein the domain is expressed as CL. The VH and VL regions may be divided into hypervariable domains called complementarity-determining regions (CDR), wherein more conservative regions called framework region (FR) are included in VH and VL regions. Each of VH and VL consists of three CDRs and four FRs, wherein the CDRs and FRs are arranged in the following order from an amino-terminal to a carboxyl-terminal: FR1, CDR1, FR2, CDR2, FR3, CDR3, and FR4. A immunoglobulin molecule may be an arbitrary type (for example, IgE, IgM, IgD, IgA, and IgY), class (for example, IgG 1, IgG2, IgG 3, IgG4, IgA1, and IgA2) or a subclass.

The term “Fc region” is used herein to define a C-terminal region of an immunoglobulin heavy chain that may be generated by a papain-degradation of a complete antibody. An Fc region may be an original sequence Fc region or a variant Fc region. An Fc region of an immunoglobulin generally includes two constant domains, CH2 domain, and CH3 domain, and, arbitrarily, CH4 domain.

A binding protein includes one or more “antigen-binding sites,” wherein the antigen-binding site is an antibody fragment having the capacity for specific binding to an antigen, and may be specifically bound to an antigen to be bispecific, dual-specific, or multi-specific. In the present invention, an antigen-binding site includes a heavy chain variable region of VH1 or VH2 and a light chain variable region of VL1 or VL2, wherein each of the variable region respectively includes a same or different antigen-binding site.

The term “bispecific” or “dual-specific” refers to a characteristics of a binding protein that may specifically bind to two different targets to adjust the activity of the targets. A bispecific or dual-specific protein may be prepared, for example, by a bonding of monoclonal antibodies specifically binding to each target or fragments thereof. A bispecific or dual-specific protein has two distinguishable antigen-binding arms (specific to two targets) and is monovalent to each of the antigens binding to the arms.

In the present invention, the bispecificity or dual-specificity may be defined by m and n, wherein m an n are an integer between 1 and 10, for example, an integer between 1 and 6, an integer between 1 and 4, or an integer between 2 and 3. In one example of the present invention, a binding protein may be tetravalent bispecific antibody, wherein VH1 or VH2 is a heavy chain variable region respectively comprising a different antigen-binding region, VL1 or VL2 is a light chain variable region respectively comprising a different antigen-binding region, and m and n are respectively 1.

The term “linker” used herein refers to two or more amino acid residues linked by a peptide bond and used to connect one or more heavy chain variable region and/or a light chain variable region. In the present invention, a linker may or may not exist. If a linker exists, L1 is a linker existing between VH1 and VH2 to link VH1 and VH2 consecutively and L2 is a linker existing between VL1 and VL2 to link VL1 and VL2 consecutively.

In one example of the present invention, with regard to the number of L1 that is a linker existing between VH1 and VH2 in a first polypeptide including VH1-[(L1)a-VH2]m-Xb to link VH1 and VH2 consecutively, a may be 1, wherein the L1 is a linker selected from the group consisting of ASTKGP (SEQ ID NO: 1), ASTKGPSVFPLAP (SEQ ID NO: 2), and GGGGSGGGGS (SEQ ID NO: 3).

In another example of the present invention, with regard to the number of L2 that is a linker existing between VL1 and VL2 in a second polypeptide including VL1-[(L2)c-VL2]n to link VL1 and VL2 consecutively, c may be 1, wherein the L2 is a linker selected from the group consisting of TVAAP (SEQ ID NO: 4), TVAAPSVFIFPP (SEQ ID NO: 5), and GGSGGGGSG (SEQ ID NO: 6).

The term “inter-disulfide domain (IDD)” refers to a region including a “inter-disulfide bridge” to connect a heavy chain and a light chain. In the present invention IDD1 of a first polypeptide and IDD2 of a second polypeptide may or may not exist, respectively.

In one example of the present invention, a heavy chain may include an IDD between VH2 and Xb to generate an inter-disulfide bridge with a light chain, wherein the IDD may be expressed as IDD1 or, in some cases, as a first polypeptide consisting of VH1-[(L1)a-VH2-(IDD1)b]m-Xc. The IDD1 may be, for example, EPKSC (SEQ ID NO: 7).

In another example of the present invention, a light chain may include an IDD at an C-terminal to generate an inter-disulfide bridge with a heavy chain, wherein the IDD may be expressed as IDD2 or, in some cases, as a second polypeptide consisting of VL1-[(L2)d-VL2-(IDD2)e]n. The IDD2 may be, for example, RGEC (SEQ ID NO: 8).

Since the IDD (IDD1 and/or IDD2) may or may not exist in a first polypeptide and or a second polypeptide, respectively, a first polypeptide including IDD1 may bind to a second polypeptide including IDD2 or a first polypeptide including IDD1 may bind to a second polypeptide not including IDD2. In addition, a first polypeptide not including IDD1 may bind to a second polypeptide including IDD2 or a first polypeptide not including IDD1 may bind to a second polypeptide not including IDD2. The term “monoclonal antibody” used herein refers to an antibody obtained from a substantially homogenous antibody group, in other words, same antibodies excluding naturally generated mutations, wherein individual antibodies may exist in a tiny quantity in the naturally generated mutations. Since a monoclonal antibody highly specific, and is induced against a single antigen-binding site. In contrast to common (polyclonal) antibody agents including different antibodies typically directed to different determinants (epitopes), each monoclonal antibody is directed to a single determinant on an antigen. The term “epitope” used herein refers to a protein determinant to which an antibody may specifically bind. An epitope generally consists of a surface molecular group, for example, an amino acid or sugar side chain, and has not only a specific three-dimensional structural characteristic but also a specific charge characteristic. A conformational epitope and a non-conformational epitope are distinguished by the fact that the binding to electron of the former is lost in the presence of a denaturing solvent but that of the latter is not lost.

A non-human (for example, murine) antibody of a “humanized” type refers to a chimeric antibody containing a minimum sequence derived from a non-human immunoglobulin. In most cases, a humanized antibody is a human immunoglobulin (receptor antibody) prepared by substituting residues of receptor hypervariable region with residues of hypervariable region of a non-human species (donor antibody) having intended specificity, affinity, and capacity, for example, mouse, rat, rabbit or a non-human primate.

The term “human antibody” used herein refers to a molecule derived from a human immunoglobulin, wherein entire amino acid sequence constituting an antibody, such as a complementarity-determining region and a structural region, consists of a human immunoglobulin.

The term “variable region” used herein refers to a light chain and heavy chain part of an antibody molecule including amino acid sequences of a complementarity-determining region (CDR; such as CDR1, CDR2, and CDR3) and a framework region (FR).

The term “complementarity-determining region” (CDR; such as CDR1, CDR2, and CDR3) refers to amino acid residues of an antibody variable region, wherein the variable region is needed for binding to an antigen. Each variable region typically has three CDRs identified as CDR1, CDR2, and CDR3. The term “framework region” (FR) used herein refers to amino acid residues except the CDR residues. Each variable region typically has four FRs identified as FR1, FR2, FR3, and FR4.

The term “variant” used herein refers to a mutation of amino acid sequence constituting a heavy chain variable region and/or a light chain variable region, for example, substitution, addition, and/or deletion, and may include an arbitrary mutation without limitation unless antigen-binding and efficacy are inhibited. The introduction of a mutation to a binding protein of the present invention may be applied to, for example, an external variable region or an internal variable region, or both an external variable region and an internal variable region.

In one example of the present invention, a binding protein of the present invention may include a variant prepared by substituting one or more amino acids selected from the group consisting of FR2 H44, FR2 H46, FR4 H101, and FR4 H103 in the VH1 and/or VH2 with cysteine. For example, in the sequence of WVRQAPGKGLEWVS corresponding to a heavy chain FR2 of SEQ ID NO: 9, the ninth amino acid, glycine (G) and the 11th amino acid, glutamic acid (E) are H44 and H46, respectively, and may be substituted with cysteine, respectively. In addition, FR4 H101 and FR4 H103 sites of a heavy chain variable region are the sites to which an inter-disulfide bridge may be introduced, and the 98th alanine (A) or the 100th glycine (G) in SEQ ID NO: 9 may be substituted with cysteine, respectively. In another example of the present invention, a binding protein may include a variant prepared by substituting with cysteine one or more amino acids selected from the group consisting of FR4 L100, FR4 L98, FR2 L44, FR2 L42, and FR2 L43 in the VL1 and/or VL2. For example, in the sequence of LAWYQQKPGKAPKLLIY corresponding to a light chain FR2 of SEQ ID NO: 10, the tenth amino acid, lysine (K), the 11th amino acid, alanine (A), and the 12th amino acid, proline (P), are L42, L43, and L44, respectively, and may be substituted with cysteine, respectively. In the cases of L100 and L98, in the sequence of FGQGTKVEIK corresponding to a light chain FR4 of SEQ ID NO: 10, the first phenylalanine (F) and glutamine (Q) are L98 and L100, respectively, may be substituted with cysteine, respectively.

In one example of the present invention, a combination of H44-L100 among the light chain or heavy chain cysteine substitution sties was selected as an engineering site, because the distance between two carbons at the H44-L100 site was as close as about 5A and thus was considered as a site most appropriate for engineering through cysteine substitution (Zhao, J.-X., Yang, L., Gu, Z.-N., Chen, H.-Q., Tian, F.-W., Chen, Y.-Q., Zhang, H., and Chen, W. (2011). Stabilization of the Single-Chain Fragment Variable by an Interdomain Disulfide Bond and Its Effect on Antibody Affinity. Ijms 12, 1-11.)

The binding protein of the present invention may bind to targets of ABCF1; ACVR1; ACVR1B; ACVR2; ACVR2B; ACVRL1; ADORA2A; aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (kathepsin B); CX3CL1 (SCYD1); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB2IP; DES; DKFZp451J0118; DNCL1; DPP4; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (gelsolin); GSTP1; HAVCR2; HDAC4; HDAC5; HDAC7A; HDAC9; HGF; HIF1A; HIP1; histamine and histamine receptor; HLA-A; HLA-DRA; HM74; HMOX1; HUMCYT2A; ICEBERG; ICOSL; ID2; IFN-a; IFNA1; IFNA2; IFNA4; IFNA5; IFNA6; IFNA7; IFNB1; IFN-γ; IFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2;IL1RL1; IL1RL2 IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (keratin 19); KRT2A; KRTHB6 (hair specific type II keratine); LAMAS; LEP (leptin); Lingo-p75; Lingo-Troy; LPS; LTA (TNF-β); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionein-III); MTSS1; MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p21Rac2); RARB; RGS1; RGS13; RGS3; RNF110 (ZNF144); ROBO2; S100A2; SCGB1D2 (lipophilin B); SCGB2A1 (mammaglobin 2); SCGB2A2 (mammaglobin 1); SCYE1 (endothelial monocyte-activation cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Spr1); ST6GAL1; STAB1; STAT6; STEAP; STEAP2; TB4R2; TBX21; TCP10; TDGF1; TEK; TGFA; TGFB1; TGFB1I1; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-α; TNFAIP2 (B94); TNFAIP3; TNFRSF11A; TNFRSF1A; TNFRSF1B; TNFRSF21; TNFRSFS; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; toll type receptor; TOP2A (Topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2 (SCM-1b); XCR1 (GPRS/CCXCR1); YY1; or ZFPM2.

If mutations having biological equivalent activity are considered, a polypeptide, a binding protein or a nucleic acid encoding the same is interpreted in the present study to include sequences showing substantial identity with the sequences disclosed in SEQ ID NOs. The substantially identical sequences refer to sequences showing preferably at least 61%, more preferably at least 70%, still more preferably at least 80%, most preferably at least 90%, 91%, 92%, 93%, 94%, 95%, 96%, 97%, 98%, or 99% of homology to the sequences of the present invention as measured using one of the sequence comparison algorithms known to those ordinarily skilled in the art, by which the sequence of the present invention is maximally aligned to another arbitrary sequence. Methods of alignment of sequences for comparison are well-known in the art. NCBI Basic Local Alignment Search Tool (BLAST) is available from several sources, including the National Center for Biological Information (NCBI), and may be used on the Internet in connection with sequence analysis programs, such as blastp, blastn, blastx, tblastn and tblastx. BLAST may be accessed at http://www.ncbi.nlm.nih.gov/BLAST/. A description of a method of comparing sequence identity using this program is available at http://www.ncbi.nlm.nih.gov/BI-AST/blast help.html.

A sequence of the present invention may include sequences 60%, 70%, 75%, 80%, 85%, 90%, 91%, 92%, 93%, 94%, 95%, 96%, 97%, 98%, 99%, or higher homology with a sequence disclosed herein or a whole sequence. The homology may be determined by sequence comparison and/or alignment by methods well-known in the art. For example, a sequence comparison algorithm (i.e., BLAST or BLAST 2.0), manual alignment, or a visual test may be used to determine the percentage sequence homology of the nucleic acid or protein of the present invention.

Another aspect of the present invention relates to a binding protein comprising a first polypeptide consisting of VH1-[(L1)a-VH2-(IDD1)b]m-Xc and a second polypeptide consisting of VL1-[(L2)d-VL2-(IDD2)e]n, wherein VH1 or VH2 of the first polypeptide is a heavy chain variable region respectively including a same or different antigen-binding region or a variant thereof, VL1 or VL2 of the second polypeptide is a light chain variable region respectively including a same or different antigen-binding region or a variant thereof, L1 of the first polypeptide is a linker existing between VH1 and VH2 to link VH1 and VH2 consecutively, L2 of the second polypeptide is a linker existing between VL1 and VL2 to link VL1 and VL2 consecutively, IDD1 of the first polypeptide is an inter-disulfide domain for generating a disulfide bridge to a light chain, IDD2 of the second polypeptide is an inter-disulfide domain for generating a disulfide bridge to a heavy chain, and X of the first polypeptide is a Fc including CH2 and CH3 domains, a, b, c, d, and e are respectively 0 or 1, and m and n are respectively an integer between 1 and 10. The specific description and examples with regard to the conformation described above may be equally applied to the invention related to the binding protein.

Another aspect of the present invention relates to an antibody comprising a plurality of binding proteins. The plurality of binding proteins may be bound, for example, by i) a bonding based on the interaction between wild type binding proteins, wherein the interaction includes various interactions known in the art, such as protein bonding such as leucine zipper of JUN domain and FOS domain or noncovalent interaction, manipulated CH domain, and manipulated mutual bonding surface, ii) disulfide bonding by introduction of cysteine, and iii) coiled-coil bonding by coiled-coil domain fusion, and other bonding methods known in the art may be included without limitation.

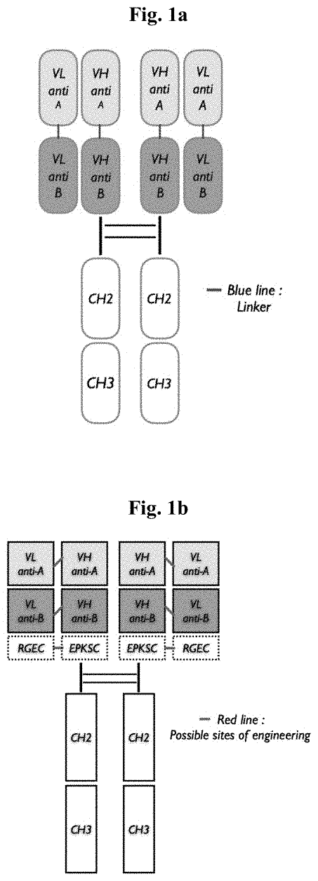

Two or more, three or more, or four or more of the binding proteins may be included, and the plurality of binding protein may have, for example, a type wherein two binding proteins including a first polypeptide consisting of VH1-[(L1)a-VH2-(IDD1)b]m-Xc and a second polypeptide consisting of VL1-[(L2)d-VL2-(IDD2)e]n are linked through an inter-disulfide bridge (FIGS. 1a and 1b).

Another aspect of the present invention relates to a conjugates comprising the binding proteins.

The term “conjugate” used herein refers to a binding protein chemically binding to a compound, a toxin, or others, for example, a therapeutic agent or a cytotoxic agent. A binding protein may be bound to a chemical compound, a mixture of chemical compounds, a biological macromolecule, or an extract prepared from a biological material, and, as a therapeutic agent or a cytotoxic agent, a toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and an analogue or a homolog thereof, but not limited thereto.

In one example of the present invention, a binding protein may be an H₂L₂ type multivalent antibody, wherein a first polypeptide including IDD1 is bound to a second polypeptide including and/or not including IDD2 and a cysteine-specific conjugation is formed with a free cysteine in EPKSC (SEQ ID NO: 7) included as IDD1 in the first polypeptide, for example, a compound, a toxin, for example, a therapeutic agent or a cytotoxic agent, or PEG (PEGylation) may be bound to the binding protein.

The present invention also relates to a nucleic acid encoding a first polypeptide and/or a second polypeptide of the binding protein.

The nucleic acid may be separated to recombinantly produce a first polypeptide and/or a second polypeptide of a binding protein. The nucleic acid is separated, and the separated nucleic acid is inserted to a replicable vector to perform additional cloning (DNA amplification) or additional expression. On the basis of this, another aspect of the present invention relates to a vector including the nucleic acid.

The term “nucleic acid” used herein comprehensively refers to DNA (gDNA and cDNA) and RNA molecules, and a nucleotide that is a basic unit of a nucleic acid includes not only natural nucleotides but also analogues, wherein a sugar or base part is modified in the analogues. A sequence of the nucleic acid of the present invention may be modified. The modification includes addition, deletion, or nonconservative substitution or conservative substitution of a nucleotide.

A DNA encoding a first polypeptide and/or a second polypeptide of a binding protein may be easily separated or synthesized by using a commonly known process with the DNA (for example, by using an oligonucleotide probe that may specifically bind to a DNA). Many vectors are available. A vector component generally includes one or more of the following, but is not limited thereto: a signal sequence, a replication origin, one or more marker gene, an enhancer factor, a promoter, and a transcription termination sequence.

The term “vector” used herein refers to a means to express a target gene in a host cell and includes a plasmid vector; a cosmid vector; and a virus vector, such as a bacteriophage vector, an adenovirus vector, a retrovirus vector, and an adeno-associated virus. In the vector, a nucleic acid encoding an antibody is operatively linked with a promoter.

The term “operatively linked” used herein means a functional bonding between a nucleic acid expression regulatory sequence (e.g.: a promoter, a signal sequence, or an array at a transcription regulator binding site) and another nucleic acid sequence, wherein the regulatory sequence regulates transcription and/or translation of the other nucleic acid sequence through the functional bonding.

In a case a prokaryote is used as a host, a vector generally includes a powerful promoter that may promote transcription (for example tac promoter, lac promoter, lacUV5 promoter, lpp promoter, pLλ promoter, pRλ promoter, rac5 promoter, amp promoter, recA promoter, SP6 promoter, trp promoter, and T7 promoter, etc.), a ribosome-binding site, and a transcription/translation termination. In addition, for example, a eukaryote is used as a host, a vector generally includes a promoter derived from a genome of a mammalian cell (example: methallothionein promoter, β-actin promoter, human hemoglobin promoter, and human muscle creatine promoter), or a promoter derived from a mammalian virus (example: adenovirus late promoter, vaccinia virus 7.5K promoter, SV40 promoter, cytomegalovirus (CMV) promoter, HSV tk promote, mouse mammary tumor virus (MMTV) promoter, HIV LTR promoter, moloney virus promoter, Epstein-Barr virus (EBV) promoter, and Rous sarcoma virus (RSV) promoter), and a polyadenylation sequence is generally included as a transcription termination sequence. In some cases, a vector may be fused with another sequence to make easy purification of a binding protein and/pr a polypeptide expressed therefrom. A fused sequence may be, for example, glutathione S transferase (Pharmacia, USA), a maltose binding protein (NEB, USA), FLAG(IBI, USA), 6×His (hexahistidine; Quiagen, USA), and others.

The vector includes antibiotic-tolerant genes commonly used as a selectable marker in the art, for example, genes tolerant to ampicillin, gentamicin, carbenicillin, chloramphenicol, streptomycin, kanamycin, geneticin, neomycin, and tetracycline.

Another aspect of the present invention relates to a cell transformed by using the vector. A cell used to generate a binding protein and/or a polypeptide of the present invention may be a prokaryote, a yeast, or a higher eukaryote cell, but not limited thereto.

Prokaryote host cells, such as Escherichia coli, Bacillus strains, such as Bacillus subtilis and Bacillus thuringiensis, Streptomyces, Pseudomonas (for example, Pseudomonas putida), Proteus mirabilis, and Staphylococcus (for example, Staphylocus carnosus) may be used.

Most attention is paid to animal cells, and an example of useful host cell strains may be COS-7, BHK, CHO, CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, 3T3, RIN, A549, PC12, K562, PER.C6, SP2/0, NS-0, U20S, or HT1080, but not limited thereto.

Another aspect of the present invention relates to a method of preparing a binding protein and/or a polypeptide, wherein the method includes (a) culturing of the cell; and (b) recovering the binding protein and/or the polypeptide from the cultured cell.

The cell may be cultured in various media. Commercially available media may be used as culture media without limitation. Any other necessary supplements known to those skilled in the art may also be included at appropriate concentrations. The culture conditions, such as temperature, pH, and the like, have already been used with the host cell selected for expression, and will be obvious to those skilled in the art.

Recovery of the binding protein and/or the polypeptide may be performed, for example, by removing impurities by centrifugation or ultrafiltration and by purifying the result by using affinity chromatography. Other additional purification technologies, for example, anion or cation exchange chromatography, hydrophobic interaction chromatography, and hydroxyapatite chromatography, may be used.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1. Structure of New Bispecific Antibody Format (Fv2mab)

Fv2mab that is a bispecific antibody has a tandem structure of fragments of variable domains (Fv) formed by a bonding between heavy chain variable regions (VH) and light chain variable regions (VL) of different antibodies of two or more kinds. Two or more kinds of variable regions linked as different tandems respectively have the capability of binding to different antigens independently or simultaneously, and has a form where heavy chain CH1 domain, kappa or lambda, and light chain CL domain are removed (FIG. 1a).

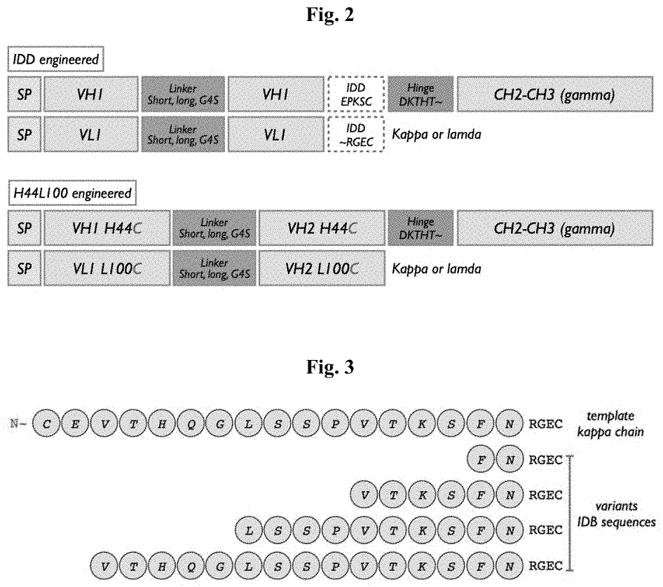

To secure binding affinity to individual antigens corresponding to two tandem-linked variable domains (Fv), a short linker (VH: ASTKGP SEQ; ID NO: 1, VL: TVAAP; SEQ ID NO: 4), a long linker (VH: ASTKGPSVFPLAP; SEQ ID NO: 2, VL: TVAAPSVFIFPP; SEQ ID NO: 5), and a G4S linker (VH: GGGGSGGGGS; SEQ ID NO: 3, VL: GGSGGGGSG; SEQ ID NO: 6) was used between an outer variable domain and an inner variable domain to secure structural independence of individual variable domains (FIG. 2).

A parental antibody used as proof of concept (POC) has proved the potential and superiority of Fv2mab by using anti-VEGFA, anti-TNF-α, and anti-IL17A monoclonal antibody as a template.

### Example 2. Fv2mab Engineering for Thermodynamic Stability

The structure of an Fv2mab bispecific antibody is an H2L2 type multivalent tetramer having two or more consecutive Fvs (VH and VL). To exist in a structure of tetramer (H2L2), a heavy chain CH domain and a light chain CL domain of a general antibody are linked by a inter disulfide bridge (covalent bond). However, in the case of Fv2mab where CH1 and CL are deleted, the structure and stability are determined by the binding force (hydrophobic bonding, ionic bonding, hydrogen bonding, etc.) between VH and VL of the tandem-linked variable domains (Fv), and, in contrast to a general antibody, an intra-disulfide bridge (covalent bond) does not exist between a heavy chain and a light chain. Therefore, the binding force determining the structure of a heavy chain and a light chain at a low concentration is expected to be lower than that of general antibodies, and, consequently, the pK/pD is also expected to be lower than that of general antibodies.

Therefore, the overall thermodynamic stability of Fv2mab was increased by substituting an amino acid at an appropriate site where VH and VL interacts with each other and by using an additional method to introduce a cysteine disulfide bridge.

In addition, another method was applied to introduce a sequence including the light chain C-terminal RGEC amino acids (SEQ ID NO: 8) linked with the heavy chain upper hinge (EPKSC; SEQ ID NO: 7) through an inter-disulfide bridge by elongating the length of the sequence with four amino acids each time (FIG. 3). An external variable region or an internal variable region, or both variable regions may be applicable as a site for introducing a cysteine disulfide bridge by methods of substitution and addition. The sites of introducing an inter-disulfide bridge by substitution are shown in Table 1, and the structure formed by introducing an inter-disulfide bridge is shown in FIG. 1b.

TABLE 1
Sites where disulfide bridge engineering may be performed.

| Site pair | Locations | Distance (Å) |
|---|---|---|
| H44-L100 | FR H2, FR L4 | 5.36 |
| H46-L98 | FR H2, FR L4 | 6.23 |
| H101-L44 | FR H4, FR L2 | 6.58 |
| H103-L42 | FR H4, FR L2 | 6.98 |
| H103-L43 | FR H4, FR L2 | 5.73 |

### Example 3. Preparation of Fv2mabs

A parental antibody used for the preparation of various combinations of bispecific Fv2mab for proof of concept (POC) was prepared by using sequences of a heavy chain variable region and a light chain variable region of HUMIRA (adalimumab, AbbVie), KOSENTYX (secukinumab, Novartis), anti-VEGFA (In-house antibody), and ABT-122 (DVD-Ig, AbbVie). A pFE expression vector prepared by the present applicants was used as an animal cell expression vector for the production of recombinant proteins of bispecific antibody Fv2mab. A heavy chain variable region and a light chain variable region of an antibody consist of a hypervariable region (CDR) and a framework (FR). Since FR1 that is a starting point and FR4 that is end point in a variable region consist of similar amino acid sequences in most antibodies, connecting two kinds of variable regions in tandem by a method such as PCR is difficult. This is because preparing and using specifically binding primers are difficult due to the similar nucleotide sequences of FR1 and FR4. Therefore, Fv2mab series were prepared by using a three-piece ligation method in a combination, such as VH1+VH2+pFE vector or VL1+VL2+pFE vector, wherein PCR is performed respectively with a heavy chain variable region and a light chain variable region in a DNA template of a parental antibody.

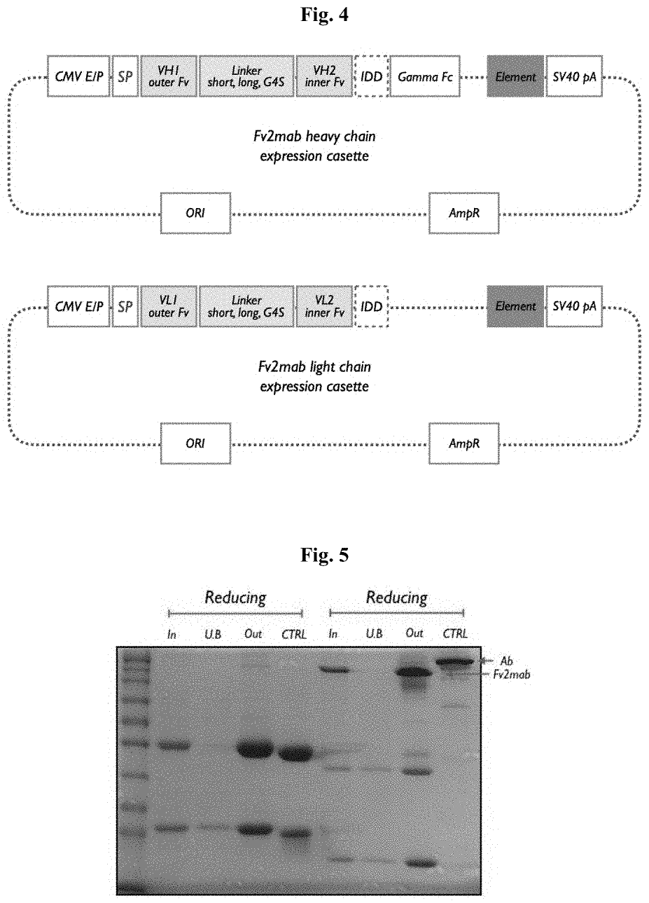

Tandem-linked VH1 and VH2 and tandem-linked VL1 and VL2 may be linked by using a short linker, a long linker or a G4S linker depending on characteristics thereof, and a cysteine disulfide bridge may be introduced to an appropriate site to improve the thermodynamic stability (FIG. 4). In the present invention, in the cases of engineered (cysteine disulfide bridge) Fv2mab, ‘e’ was added in front of a variable region to which engineering was introduced for notation (example: A cysteine disulfide bridge was introduced to anti-TNF-α Fv in the case of eTNF-IL17, and to anti-IL17A Fv in the case of TNF-eIL17.).

In addition, the engineering performed by introducing an IDD (RGEC) at an C-terminal of a light chain was denoted by adding “RGEC” (example: VEGF-TNF-RGEC). Type II restriction enzyme sapI (NEB, England) was used in three-piece ligation cloning, and T4 DNA ligase (NEB, England) was used for ligation of DNA fragments. Table 2 shows the Fv2mab series prepared for the present invention. Table 3 shows the sequence of each Fv2mab series.

TABLE 2
Fv2mab clone series

| | Outer paratope/ | Inner paratope/ | | |
|---|---|---|---|---|
| Fv2mab clones | parent Ab | parent Ab | Linker | Disulfide bridge |
| VEGF-TNF-SL- | anti-VEGFA/ | anti-TNF-a/ | Short linker | RGEC |
| RGEC | F6 (in-house) | HUMIRA | | |
| VEGF-TNF-SL- | anti-VEGFA/ | anti-TNF-a/ | Short linker | RGE |
| ΔC | F6 (in-house) | HUMIRA | | no inter S-S bridge |
| TNF-IL17-SL | anti-TNF-a/ | anti-IL17a/ | Short linker | no inter S-S bridge |
| | HUMIRA | Secukinumab | | |
| eTNF-IL17-SL | anti-TNF-a/ | anti-IL17a/ | Short linker | H44L100 |
| | HUMIRA | Secukinumab | | |
| eTNF-IL17-LL | anti-TNF-a/ | anti-IL17a/ | Long linker | H44L100 |
| | HUMIRA | Secukinumab | | |
| eTNF-IL17-G4S | anti-TNF-a/ | anti-IL17a/ | G4S linker | H44L100 |
| | HUMIRA | Secukinumab | | |
| TNF-eIL17-G4S | anti-TNF-a/ | anti-IL17a/ | G4S linker | H44L100 |
| | HUMIRA | Secukinumab | | |
| eABT122 | anti-TNF-a/ | anti-IL17a/ | G4S linker | H44L100 |
| | HUMIRA | AbbVie ABT122 | | outer variable |
| | | | | region |

TABLE 3
Sequence of Fv2mab series

| Fv2mab series | first polypeptide | second polypeptide |
|---|---|---|
| VEGF-TNF-SL-RGEC | SEQ ID NO: 9 | SEQ ID NO: 11 |
| | (VEGF-TNF-SL-RGEC-Fc: SEQ | |
| | ID NO: 10) | |
| VEGF-TNF-SL-ΔC | SEQ ID NO: 12 | SEQ ID NO: 14 |
| | (VEGF-TNF-SL-ΔC-Fc: SEQ ID NO: 13) | |
| TNF-IL17-SL | SEQ ID NO: 15 | SEQ ID NO: 17 |
| | (TNF-IL17-SL-Fc: SEQ ID NO: 16) | |
| eTNF-IL17-SL | SEQ ID NO: 18 | SEQ ID NO: 20 |
| | (eTNF-IL17-SL-Fc: SEQ ID NO: 19) | |
| eTNF-IL17-LL | SEQ ID NO: 21 | SEQ ID NO: 23 |
| | (eTNF-IL17-LL-Fc: SEQ ID NO: 22) | |
| eTNF-IL17-G4S | SEQ ID NO: 24 | SEQ ID NO: 26 |
| | (eTNF-IL17-G4S-Fc: SEQ ID NO: 25) | |
| TNF-eIL17-G4S | SEQ ID NO: 27 | SEQ ID NO: 29 |
| | (TNF-eIL17-G4S-Fc: SEQ ID NO: 28) | |
| eABT122_Fv2mab | SEQ ID NO: 30 | SEQ ID NO: 32 |
| | (eABT122_Fv2mab-Fc: SEQ ID | |
| | NO: 31) | |

### Example 4. Fv2mab Expression and Purification Through Protein A

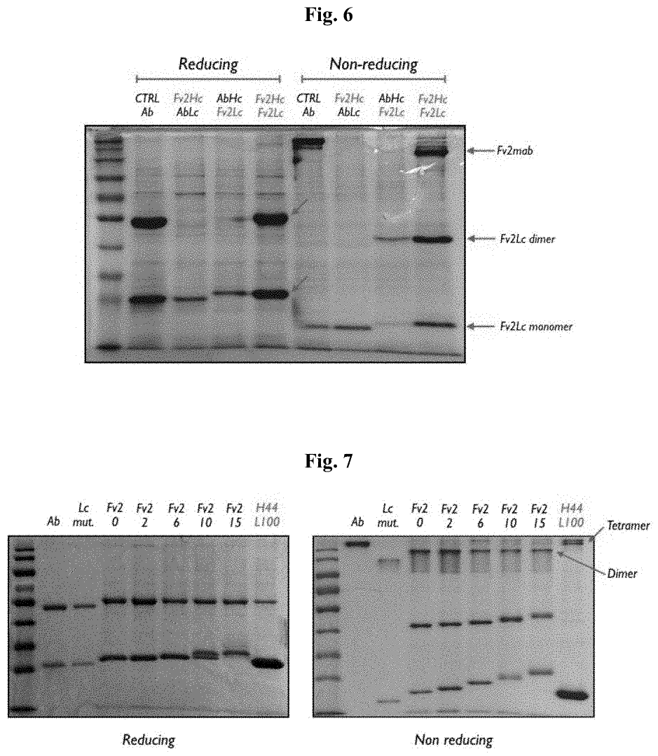

In the expression verification and production of Fv2mab clone series recombinant proteins, transient expression system was used, and HEK-293F strain (Invitrogen, USA) was used as a host cell. Expression of Fv2mab was performed by a method of co-transfection, wherein the co-transfection was performed at 1:1 w/w ratio of Fv2mab-Hc DNA to Fv2mab-Lc DNA. Polyethylenimine (PEI) was used as a transfection agent for transfection by preparing a polyplex at a ratio of DNA:PEI=1:4 (w/w). On the fifth day after transfection, the batch culture was discontinued, the expression medium was harvested, and the antibody was pulled down from the medium by using protein A beads (GE healthcare, USA). The degree and conformation of expression was verified by using SDS-PAGE/Coomassie Blue staining for each fraction of input (expression medium), unbound sample (flowthrough), and output (bound Fv2mab protein) (FIG. 5).

A parental IgG antibody was used as a control group sample of the pull down assay. As described above, a series of Fv2mab antibodies was cloned to the pFE expression vector, transfected to HEK 293F cells by using PEI, and produced from the cells by batch culture. Six to seven days after the transfection when the cell survival rate is measured to be about 60% to 70%, the batch culture was discontinued, and the expression medium was centrifugated (4,800 rpm, 30 min, 4° C.) to remove the debris. Then, the supernatant was filtered by using a 0.22 μm TOP-filter (Millipore, USA). Subsequently, the filtered supernatant including Fv2mab underwent an affinity chromatography purification process using protein A beads (GE healthcare, USA), followed by dialysis with pH 7.4 PBS by using Slide-A Lyzer Dialysis Cassette (Thermo, USA) for elution buffer change. The level of Fv2mab expression using HEK293F was found to be about 100 to 200 mg/L, and the pull down assay using protein A showed that most of Fv2mab expressed in the medium was recovered.

### Example 5. Structure Screening and Disulfide Bridge Engineering

Assembly of bispecific antibody Fv2mab is determined by folding of Fv2mab-heavy chain VH domain in the ER in a BiP-dependent manner. In Fv2mab, regions controlled by BiP are present in two tandem-linked heavy chain VH domains, and BiP and VH complex that are not bound to LC are degraded in ER. BiP bound to Fv2mab heavy chain VH is competitively released from VH by a light chain VL, and a heavy chain VH is assembled with a light chain VL to be secreted externally.

In an antibody, heavy chain domains, CH1 and VH, are bound to BiP which is released only by light chain CL and VL, respectively. Control by BiP does not occur by a method based on another folding partner (VH and CL or CH1 and VL), a failure of the control by BiP leads to the antibody degradation (FIG. 6).

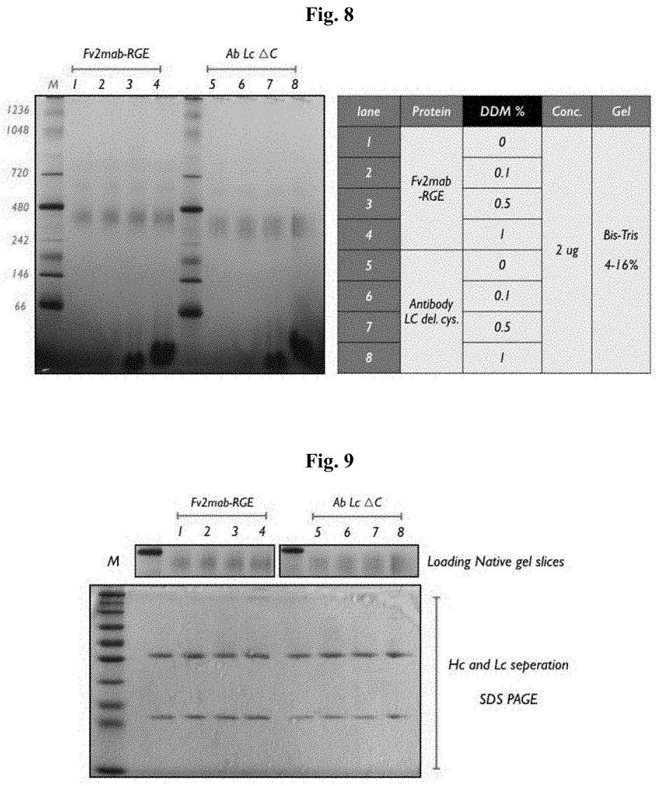

To substitute the thermodynamic stability of an inter-disulfide bridge between CH1 and CL existing in a general antibody, a method of applying a variant IDD to a light chain C-terminal including an RGEC region that could form an inter-disulfide bridge and a method of substituting with cysteine a key amino acid of VH and VL (H44L100) forming a variable region (Fv) were applied in the case of Fv2mab. The assembly, conformation, and expression pattern of the engineered Fv2mab clones were verified through SDS-PAGE after protein expression using HEK 293 (FIG. 7). The SDS-PAGE results showed that an H2L2 tetramer was effectively formed by an inter-disulfide bridge at an H44L100-engineered clone, and the level of expression is predicted to be about 50 to 100 mg/L.

### Example 6. Verification of Fv2mab Conformation Through Native PAGE

Blue Native PAGE (Invitrogen, USA) was used to verify the conformation (H2L2, tetramer) of Fv2mab formed only by a bonding between VH and VL of tandem-linked variable regions (Fv). The structure of Fv2mab was verified by using as a control group HUMIRA LCΔC (-RGE) formed only by a bonding between VH and VL in the absence of an inter-disulfide bridge by deleting cysteine connecting an inter-disulfide bridge between Fv2mab and a light chain C-terminal.

The native PAGE result verified that Fv2mab and HUMIRA LCΔC (-RGE) formed an H₂L₂ tetramer having a molecular weight from 240 kDa to 420 kDa under the condition of pH 7.4 PBS (FIG. 8).

In addition, a second SDS-PAGE analysis was performed by separating from the PAGE gel the Fv2mab tetramer band existing between 240 kDa and 420 kDa on the native PAGE gel, and the result verified both a heavy chain and a light chain of Fv2mab separated by an ionic detergent (SDS) (FIG. 9).

### Example 7. Fv2mab Purity after Protein a Chromatography

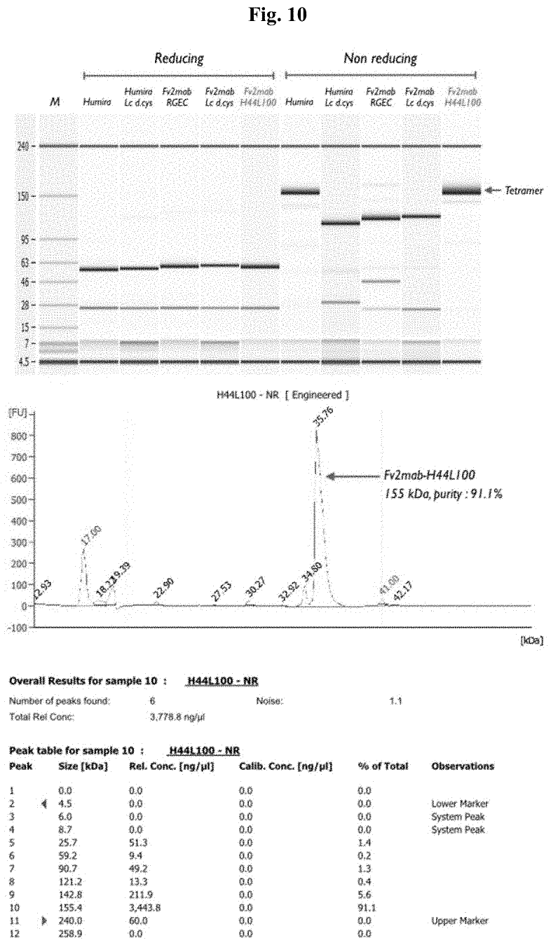

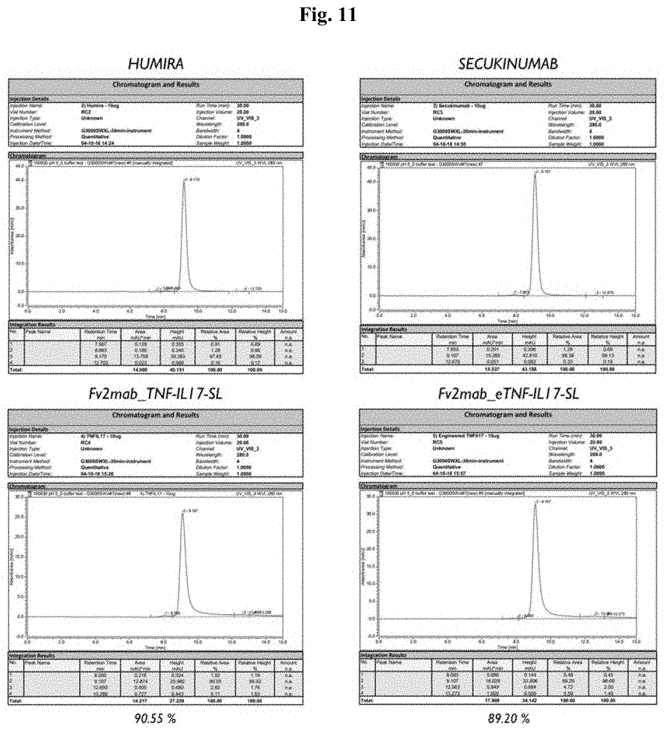

The protein purity after the expression and purification of bispecific antibody Fv2mab of various structures and combinations was analyzed by using Agilent 2100 Bioanalyzer (Agilent Technologies, Germany) and SEC-HPLC (ThermoFisher, USA). The purity analysis was performed by following the protocols provided by the manufactures with the engineered Fv2mabs prepared by introducing a cysteine disulfide bridge to increase thermodynamic stability, Fv2mabs (not engineered), and the controls of HUMIRA, HUMIRA LCΔC, and SECUKINUMAB. The result showed that an inter-disulfide bridge was formed at a considerably high ratio in the H44L100 clone in comparison with the RGEC clone in the case of cysteine disulfide bond-engineered Fv2mab clones (FIG. 10). The results obtained by using 2100 Bioanalyzer and SEC-HPLC showed that the purity of the Fv2mab recombinant proteins obtained by using the HEK293 transient expression system was about 90% or higher (FIG. 11).

### Example 8. Stability of Fv2mabs in Human Serum (In Vitro)

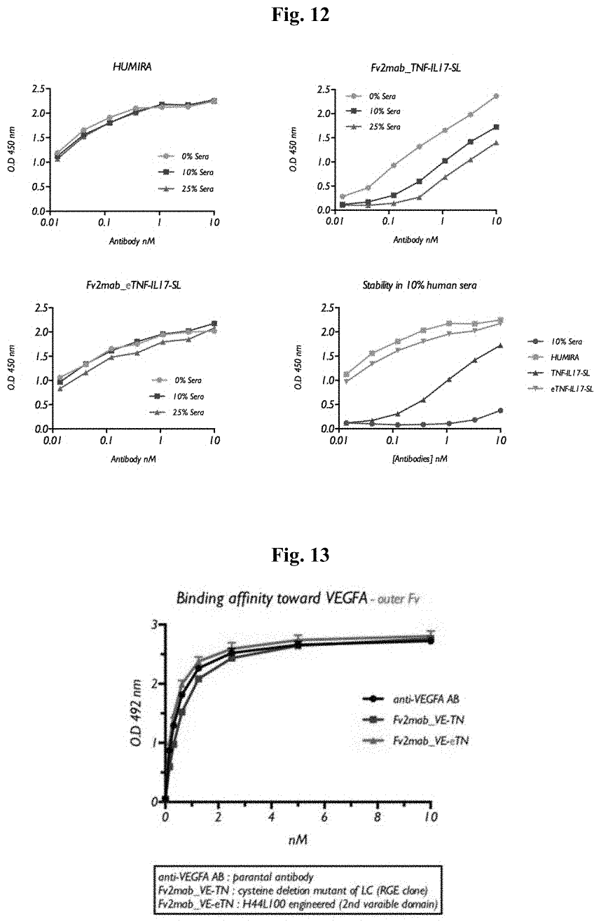

The stability of engineered Fv2mabs prepared by introducing a cysteine disulfide bridge to increase thermodynamic stability was measured in vitro by using human serum. The Fv2mab (eTNIL17) engineered by applying H44L100 substitution, Fv2mab (TNIL17) having no disulfide bridge (TNIL17), and a control antibody HUMIRA were added to 10 nM medium solutions respectively containing 0%, 5%, 10%, and 25% of human serum at 37° C. for five day for a reaction, and then the binding affinity to TNF-α was measured by ELISA to verify the stability of Fv2mab. The serum stability of the Fv2mab (eTNIL17) engineered by applying H44L100 substitution to increase the stability was equal to that of the control antibody HUMIRA, but the non-engineered Fv2mab consisting of tandem Fvs showed relatively low stability at a low concentration (FIG. 12).

### Example 9. Comparison of Binding Affinity of Fv2mab to Individual Antigens

After expressing and purifying Fv2mabs of various combinations by using the HEK293 transient expression system, the binding affinity to the IgG type parental antibodies and their antigens was compared. The binding affinity was measured by using ELISA method and OCTET system (ForteBIO, USA).

### Example 9-1. ELISA

The affinity of Fv2mabs consisting of the tandem Fv combinations of anti-TNF-α and anti-VEGFA; and anti-TNF-α and anti-IL17A was measured by using ELISA method. Antigens such as TNF-α, VEGFA, and IL-17A, were fixed on an immune-plate at a concentration of 100 ng/well (96 well) by using pH 7.4 PBS as a coating buffer through a reaction at 4° C. overnight. Each well was once washed with PBST 200 μl, and then a surface blocking reaction was performed at room temperature with 5% skim milk for one to two hours. Subsequently, after washing each well twice with PBST 200 μl, a serial dilution was performed with an appropriate bispecific Fv2mab to measure and a control parental antibody from an appropriate concentration in an interval of ½ to ⅕ for a reaction at room temperature for one to two hours. Then, each well was washed three times with PBST 200 μl to remove unbound antibodies, and a dilution was performed with horseradish peroxidase (HRP)-conjugated anti-human-Fc-HRP (Millipore, USA) at a ratio of 1:3000 for a reaction at room temperature for one hour. Then, each well was washed three times with PBST 200 μl to remove unbound detection antibodies. A color reaction of HRP was induced by using TMB solution (GEhealthcare, USA) in a volume of 100 a/well, and the reaction was terminated by using a stop solution (2.5 M H2SO4, 100 μl/well). A spectrophotometer was used to measure the absorbance at a wavelength of 450 nm to calculate the binding affinity. FIGS. 13 and 14 show the comparison of the binding affinity of anti-VEGFA antibody, Fv2mab_VE-TN, and Fv2mab_VE-eTN (H44L100 disulfide bridge engineered clone) to VEGFA and TNF-α.

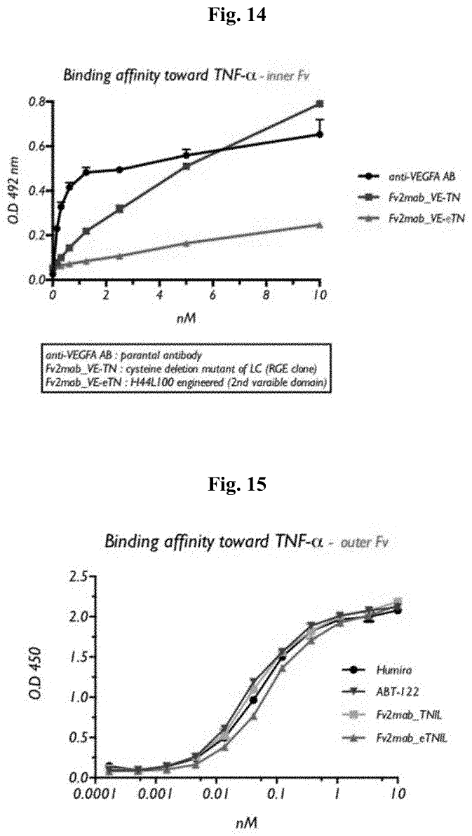

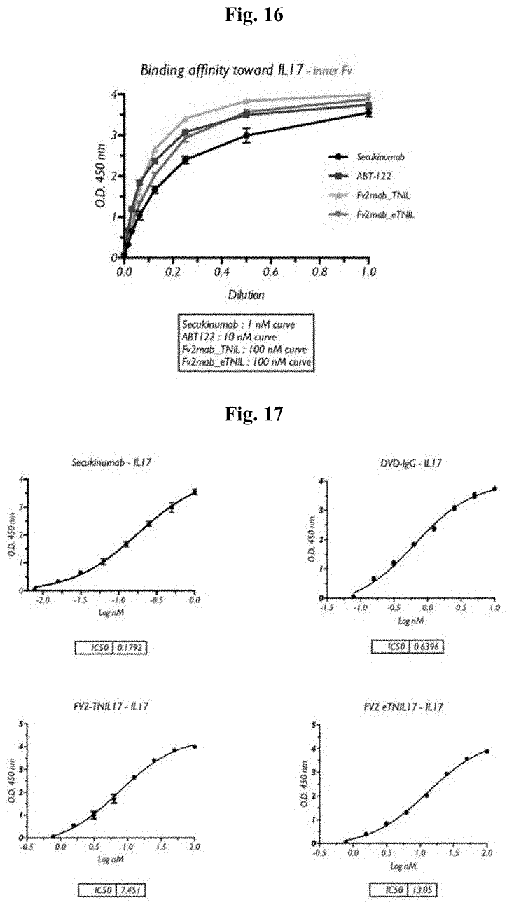

In addition, FIG. 15 shows the comparison of the binding affinity of HUMIRA, ABT-122 (DVD-Ig for neutralizing TNF-α and IL-17A, AbbVie, USA), Fv2mab_TNF-IL17, and Fv2mab_eTNF-IL17 to TNF-α, and FIGS. 16 and 17 show the comparison of the binding affinity of SECUKINUMAB, ABT-122 (AbbVie, USA), Fv2mab_TNF-IL17, and Fv2mab_eTNF-IL17 to IL-17A and the IC 50 value thereof, respectively.

### Example 9-2. OCTET (Surface Plasmon Resonance)

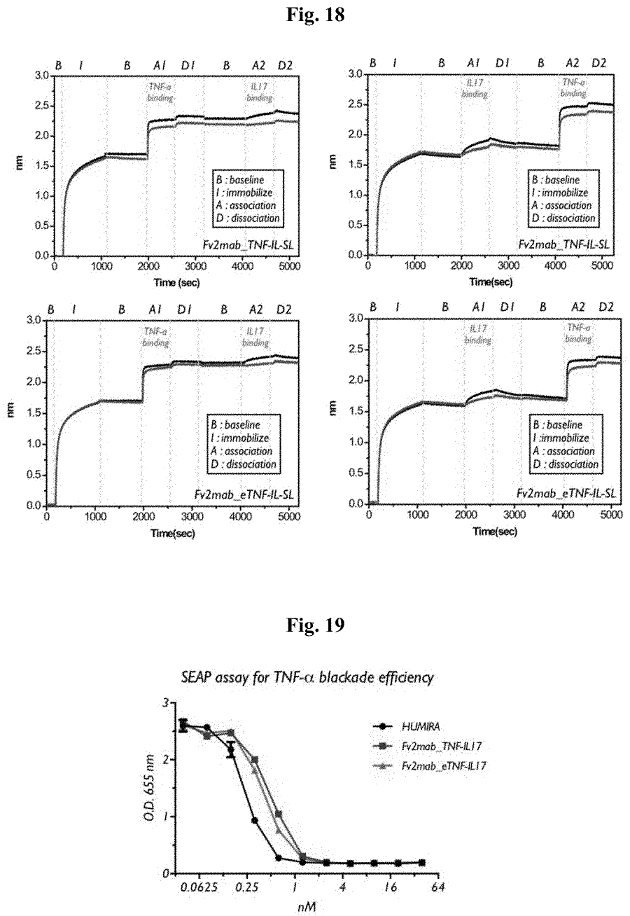

The degree and characteristic of binding of Fv2mab_TNF-IL17-SL and Fv2mab_eTNF-IL17-SL to TNF-α and IL-17 were measured by using Surface Plasmon Resonance (SPR), wherein Fv2mab_TNF-IL17-SL and Fv2mab_eTNF-IL17-SL consist of variable regions of HUMIRA and SECUKINUMAB. Measurement of label-free kinetics (protein-protein interaction) using OCTET QKe system was performed by selecting and using an anti-human IgG capture (AHC) biosensor, and Fv2mab_TNF-IL17-SL and Fv2mab_eTNF-IL17-SL including human Fc were fixed on the biosensor surface and sequentially reacted with TNF-α and IL-17 diluted with the kinetic buffer at different concentrations to collect the sensorgram over time. Table 4 shows the kinetics values plotted with the sensorgram values depending on concentrations at equilibrium. The antigen concentrations selected to obtain the kinetic values of Fv2mab_TNF-IL17-SL and Fv2mab_eTNF-IL17-SL were 1,000 nM and 333 nM for both TNF-α and IL-17, and the OCTET system was used to verify the difference of the binding force depending on the order of reacted antigens and the degree of simultaneous binding to both antigens (FIG. 18).

TABLE 4
Kinetic of Fv2mab

| | 1^{st} analytes TNF-a | | | 2^{nd} analytes IL-17 | | |
|---|---|---|---|---|---|---|
| | Kₒₙ | K_{off} | K_{D} | Kₒₙ | K_{off} | K_{D} |
| | (1/Ms) | (1/s) | (M) | (1/Ms) | (1/s) | (M) |
| Fv2mab | 4.08E+05 | <1E−07 | <1E−12 | 2.59E+03 | 5.897E−04 | 2.278E−07 |
| TNF-IL-SL | | | | | | |
| Fv2mab | 2.761E+05 | <1E−07 | <1E−12 | 6.618E+03 | 9.249E−04 | 1.398E−07 |
| eTNF-IL-SL | | | | | | |

| | 1^{st} analytes IL-17 | | | 2^{nd} analytes TNF-a | | |
|---|---|---|---|---|---|---|
| | Kₒₙ | K_{off} | K_{D} | Kₒₙ | K_{off} | K_{D} |
| | (1/Ms) | (1/s) | (M) | (1/Ms) | (1/s) | (M) |
| Fv2mab | 3.99E+03 | 3.639E−04 | 9.118E−08 | 7.11E+04 | <1E−07 | <1E−12 |
| TNF-IL-SL | | | | | | |
| Fv2mab | 9.697E+03 | 5.872E−04 | 6.056E−08 | 2.652E+05 | <1E−07 | <1E−12 |
| eTNF-IL-SL | | | | | | |

### Example 10. Blockade Efficacy of Fv2mabs (In Vitro Cell-Based Assay)

### Example 10-1. TNF-αBlockade Assay

A secreted embryonic alkalinephosphatase (SEAP) assay system using HEK blue TNF-α cell (Invivogen, USA) was used to verify the TNF-α blockade efficacy of bispecific antibody Fv2mab. The HEK Blue TNF-α cell supplied by Invivogen is a system for effective monitoring of signaling by TNF-α and includes a SEAP reporter gene at a downstream position of an NF-kB promoter regulated by signaling. A signal transmitted by TNF-α finally increases the expression of SEAP, wherein SEAP is accumulated in a culture medium and easily measured quantitatively. The quantity of SEAP accumulated in a culture medium may be measured through a coloring process by an enzymatic reaction using Quanti-Blue substrate (Invivogen, USA), and the absorbance may be measured by using a spectrophotometer to verify the blockade efficacy. The TNF-α blockade assay result showed that the efficacy of both Fv2mab_TNF-IL17-SL and Fv2mab_eTNF-IL17-SL is similar to that of HUMIRA (FIG. 19).

### Example 10-2. IL-17A Blockade Assay

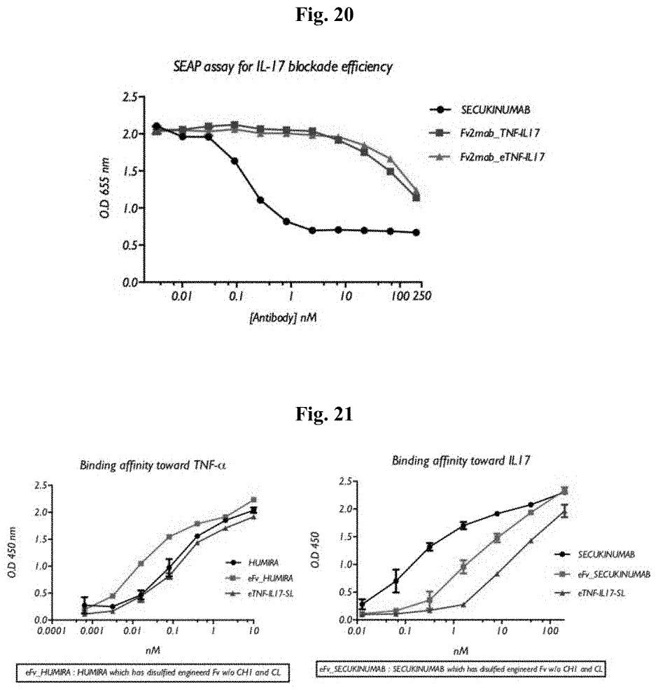

A SEAP assay system using HEK Blue IL17 cell (Invivogen, USA) was used to verify the water-soluble IL-17A blockade efficacy of bispecific antibody Fv2mab. As in the case of the HEK Blue TNF-α cell system described in Example 10-1, SEAP was used as a reporter gene also in the HEK Blue IL-17 cell system. The IL-17 blockade efficacy of Fv2mab_TNF-IL17-SL, Fv2mab_eTNF-IL17-SL, and SECUKINUMAB was quantified by an enzymatic reaction using Quanti-Blue substrate (Invivogen, USA) (FIG. 20).

### Example 11. Binding Affinity of Mono-Fv Antibody and Two Linked Fvs

The binding affinity of Fvlmab consisting of one variable region (VH1-Fc, VL1) and Fv2mab consisting of two consecutive variable regions (VH1-L-VH2-Fc, VL1-L-VL2) was compared with that of a parental antibody. The result of a transient expression of Fvlmab performed by using HEK 293 showed that the level of expression was similar to that of Fv2mab, and cysteine disulfide bridge engineering (H44L100) was applied to Fvlmab used in the present experiment for comparing binding affinity. Fvlmab was prepared by using HUMIRA and SECUKINUMAB as a parental template, and PCR was performed with the VH and VL domains of each antibody. The binding affinity of HUMIRA, Fv1mab_TNF, and Fv2mab_eTNF-IL-SL to TNF-α was compared by using ELISA, and that of SECUKINUMAB, Fvlmab_IL17, and Fv2mab_TNF-IL-SL to IL-17 was compared also by using ELISA (FIG. 21).

### Example 12. Binding Affinity Depending on Linkers

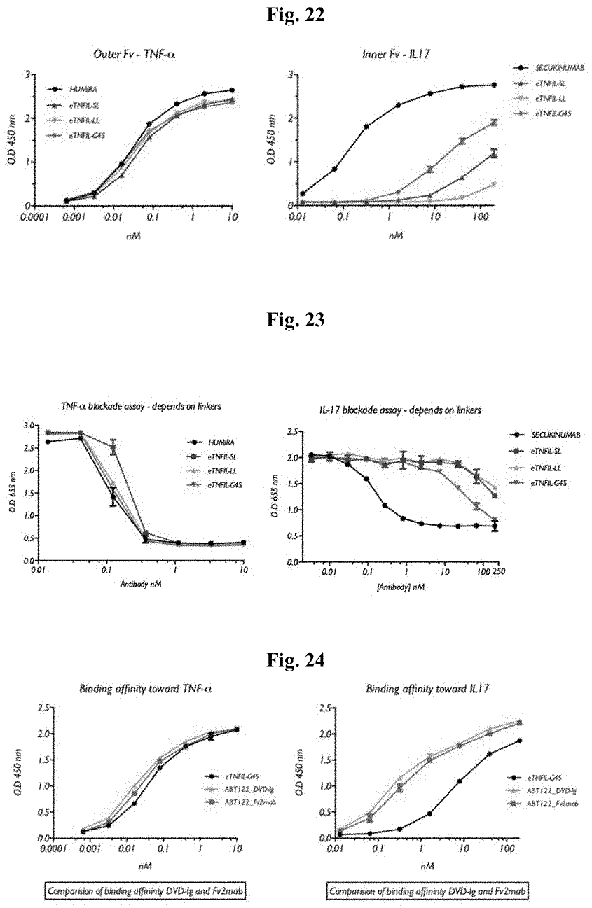

The effect of linkers connecting consecutive variable regions on antigen binding was measured by using ELISA. A short linker (VH: ASTKGP SEQ; ID NO: 1, VL: TVAAP; SEQ ID NO: 4), a long linker (VH: ASTKGPSVFPLAP; SEQ ID NO: 2, VL: TVAAPSVFIFPP; SEQ ID NO: 5), and a G4S linker (VH: GGGGSGGGGS; SEQ ID NO: 3, VL: GGSGGGGSG; SEQ ID NO: 6) were introduced between an external Fv and an internal Fv to investigate the change in binding affinity depending on linkers, and Fv2mabs including the linkers were prepared by using the HUMIRA variable region for the external Fv and the SECUKINUMAB variable region for the internal Fv. The cysteine disulfide bridge engineering of H44L100 was applied to all the three types of Fv2mabs linked with different kinds of linkers, and the binding affinity to TNF-α and IL-17 was measured by using ELISA. FIG. 22 shows the change in binding affinity of Fv2mab depending on linkers.

### Example 13. In Vitro Efficacy of Fv2mab Depending on Linkers (Cell-Based Assay)

The in vitro efficacy of Fv2mab depending on linkers linking consecutive variable regions was verified by using HEK blue TNF-α cell (Invivogen, USA) and HEK Blue IL17 cell (Invivogen, USA). The Fv2mab used in the measurement of the in vitro efficacy and prepared by introducing short, long, and G4S linkers underwent serial dilution from the concentrations of 10 nM and 200 nM, and then each of the diluted Fv2mabs underwent a reaction with TNF-α and IL-17. Quanti-Blue substrate (Invivogen, USA) was used to quantitatively verify SEAP accumulated in the culture medium, and the degree of coloring depending on the reaction was measured to verify the blockade efficacy (FIG. 23).

### Example 14. Comparison of Binding Affinity Between DVD-Ig and Fv2mab

To comparatively analyze the binding affinity depending on bispecific antibody format, ABT-122_Fv2mab having an amino acid sequence of a same variable region for ABT-122_DVD-Ig (anti-TNF-α/IL-17 bispecific antibody) and anti-TNF-α/IL-17 was prepared. Although ABT-122_DVD-Ig and ABT-122_Fv2mab have an amino acid sequence and a linker of the same variable region, the molecular weight of DVD-Ig including CH1 and CL is about 200 kDa, but that of Fv2mab without CH1 and CL is about 150 kDa. The G4S linker was used to link variable regions, and cysteine disulfide bridge engineering was introduced to ABT-122_Fv2mab. In the comparison between bispecific antibody formats, the binding affinity to TNF-α and IL-17 antigens was measured by using ELISA, and eTNFIL-G4S prepared by using the same G4S linker and a variable region of SECUKINUMAB was used as a control group for the comparative analysis. The measured affinity to TNF-α and IL-17 was the same in the two bispecific antibodies of ABT-122_DVD-Ig and ABT-122_Fv2mab having a same variable region sequence, although the bispecific antibody format was different. In addition, the molecular weight of Fv2ma may be considered as an advantage over other bispecific antibody formats in the development of antibody-based medical products, because the molecular weight of Fv2ma is about ¾ of that of DVD-Ig (FIG. 24).

### Example 15. In Vitro Efficacy (Comparison of Bispecific Antibody Format)

### Example 15-1. TNF-α and IL-17A Blockade Assay Using HEK Blue Cell

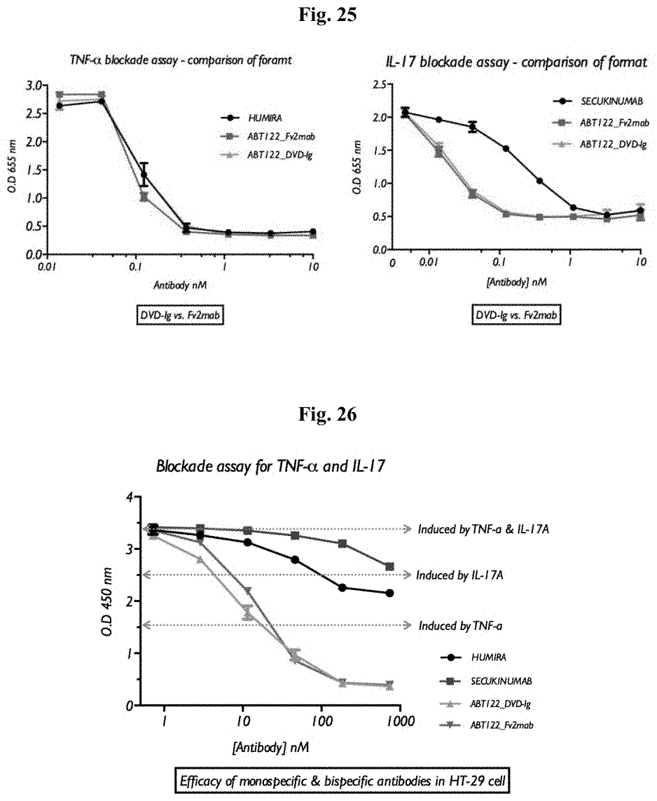

The in vitro efficacy depending on bispecific antibody format was verified by using HEK blue TNF-α cell (Invivogen, USA) and HEK Blue IL17 cell (Invivogen, USA). The degree of blocking signal transmission with respect to TNF-α and IL-17 was verified by using ABT-122_DVD-Ig and ABT-122_Fv2mab at concentrations from 10 nM in comparison with HUMIRA and SECUKINMAB (FIG. 25). The ABT-122_Fv2mab using a same variable region amino acid sequence with ABT-122_DVD-Ig showed a same degree of TNF-α and IL-17 blockade efficacy at a same molar concentration. In addition, the IL-17 blockade efficacy of ABT-122_Fv2mab was better than that of the monoclonal antibody SECUKUNUMAB. This example showed that a bispecific antibody having a same molecular weight with that of a general antibody and a significantly higher effect than that of a monoclonal antibody through the binding to two or more kinds of antigens may be selectively and easily prepared. Since the molecular weight of ABT-122_Fv2mab is about 25% less than that of DVD-Ig, it is expected that the bispecific antibody of the present invention may effectively penetrate to tissues, rapidly increase and maintain the concentration in blood, and give a higher molar effect in comparison with the weight of DVD-Ig when used in a clinical study and administered as a therapeutic agent.

### Example 15-2. TNF-α and IL-17A Blockade Assay Using HT-29 Cell

The TNF-α and IL-17A simultaneous blockade effect of the bispecific antibody was verified and compared by performing a blockade assay using HT-29 (ATCC® HTB-38™) cell. The cytokine blockade assay using HT-29 cell is performed by measuring the level of CXCL1 chemokine secreted from HT-29 cell by the signal transmitted through TNF-α, IL-17A, and IL-22. The concentrations of TNF-α, IL-17A, and IL-22 used to measure the effect of bispecific antibody of inhibiting cell signal transmission induced by cytokine in HT-29 cell were 1.17 ng/ml, 2.5 ng/ml, and 0.1 ng/ml, respectively. The medium used for HT-29 cell culture was RPMI 1640 (Hyclone, USA) medium including 10% FBS, and the medium used for cytokine blockade assay was RPMI 1640 medium including 2% FBS. The quantity of CXCL1 secreted from HT-29 was measured by using duoset ELISA kit Human CXCL1/GROa kit (R&D), and the CXCL1 measurement using ELISA was performed by following the protocol provided by the manufacturer. The bispecific antibodies (ABT122_DVD-Ig, ABT122_Fv2mab) and the monospecific antibodies (HUMIRA, SECUKINUMAB) used in the cytokine blockade assay using HT-29 were used by diluting from 736 pM to 0.7 pM in a dilution factor interval of ¼ by the same method. The level of CXCL1 induced in HT-29 cell only by 1.17 ng/ml TNF-α or only by 2.5 ng/ml IL-17A was measured by performing ELISA under same conditions and found to be about 1.5 and 2.5 O.D., respectively, and the level of CXCL1 induced by both TNF-α and IL-17A was found to be about 3.4 O.D. The degree of inhibiting cellular signal transmission simultaneously induced by both TNF-α and IL-17A in HT-29 cell was significantly higher in bispecific antibodies than in monospecific antibodies, and the bispecific antibodies ABT122_DVD-Ig and ABT122-Fv2mab showed an equivalent cytokine blockage effect at a same molar concentration (FIG. 26). This result showed that the bispecific antibody format having an antibody sequence of a same variable region may have various advantages over Fv2mab and DVD-Ig.

### INDUSTRIAL APPLICABILITY

The new format of multi-specific multivalent binding protein of the present invention may supplement drawbacks of conventional multi-specific binding proteins and inhibit target activity by simultaneously binding to two or more targets to provide excellent effects in disease treatment and diagnosis in comparison with monospecific antibody-based treatment.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A binding protein comprising a first polypeptide of VH1-[(L1)a-VH2]m-Xb and a second polypeptide of VL1-[(L2)c-VL2]n
wherein VH1 or VH2 of the first polypeptide respectively is a heavy chain variable region comprising a same or different antigen-binding region or a variant thereof,
VL1 or VL2 of the second polypeptide respectively is a light change variable region comprising a same or different antigen-binding region or a variant thereof, and the second polypeptide lacks CL,
L1 of the first polypeptide is a linker existing between VH1 and VH2 to link VH1 and VH2 consecutively,
L2 of the second polypeptide is a linker existing between VL1 and VL2 to link VL1 and VL2 consecutively,
X of the first polypeptide is a Fc comprising CH2 and CH3 domains and excluding CH1,
a, b, and c are respectively 0 or 1, and m and n are respectively an integer between 1 and 10.

2. The binding protein according to claim 1, wherein a is 1, and L1 is a linker selected from the group consisting of ASTKGP (SEQ ID NO: 1), ASTKGPSVFPLAP (SEQ ID NO: 2), and GGGGSGGGGS (SEQ ID NO: 3).

3. The binding protein according to claim 1, wherein c is 1, and L2 is a linker selected from the group consisting of TVAAP (SEQ ID NO: 4), TVAAPSVFIFPP (SEQ ID NO: 5), and GGSGGGGSG (SEQ ID NO: 6).

4. The binding protein according to claim 1, wherein the heavy chain comprises inter-disulfide bridge between VH2 and Xb to generate an inter-disulfide bridge with a light chain.

5. The binding protein according to claim 4, wherein the IDD comprises an amino acid sequence of EPKSC (SEQ ID NO: 7).

6. The binding protein according to claim 1, wherein the light chain comprises inter-disulfide bridge at an C-terminal to generate an inter-disulfide bridge with a heavy chain.

7. The binding protein according to claim 6, wherein the IDD comprises an amino acid sequence of RGEC (SEQ ID NO: 8).

8. The binding protein according to claim 1, wherein one or more amino acids selected from the group consisting of FR2 H44, FR2 H46, FR4 H101, and FR4 H103 in the VH1 and/or VH2 is substituted with cysteine.

9. The binding protein according to claim 1, wherein one or more amino acids selected from the group consisting of FR4 L100, FR4 L98, FR2 L44, FR2 L42, and FR2 L43 in the VL1 and/or VL2 is substituted with cysteine.

10. The binding protein according to claim 1, wherein the binding protein is tetravalent bispecific antibody,
wherein VH1 or VH2 respectively is a heavy chain variable region comprising a different antigen-binding region, VL1 or VL2 is a light chain variable region respectively comprising a different antigen-binding region, and m and n are respectively 1.

11. A binding protein comprising a first polypeptide consisting of VH1-[(L1)a-VH2-(IDD1)b]m-Xc and a second polypeptide consisting of VL1-[(L2)d-VL2-(IDD2)e]n,
wherein VH1 or VH2 of the first polypeptide is a heavy chain variable region respectively including a same or different antigen-binding region or a variant thereof,
VL1 or VL2 of the second polypeptide is a light change variable region respectively including a same or different antigen-binding region or a variant thereof,
L1 of the first polypeptide is a linker existing between VH1 and VH2 to link VH1 and VH2 consecutively,
L2 of the second polypeptide is a linker existing between VL1 and VL2 to link VL1 and VL2 consecutively,
IDD1 of the first polypeptide is an inter-disulfide domain for generating a disulfide bridge to a light chain,
IDD2 of the second polypeptide is an inter-disulfide domain for generating a disulfide bridge to a heavy chain, and
X of the first polypeptide is a Fc comprising CH2 and CH3 domains, a, b, c, d, and e are respectively 0 or 1, and m and n are respectively an integer between 1 and 10.

12. An antibody comprising a plurality of binding proteins according to claim 1.

13. A conjugate comprising the binding protein according to claim 1.
